(19) United States
(12) Patent Application Publication
(10) Pub. No.: US 2024/0391893 A1
(43) Pub. Date: Nov. 28, 2024

(54) COMPOSITION CONTAINING FLUORINE-CONTAINING MONOMER, METHOD FOR STABILIZING FLUORINE-CONTAINING MONOMER, METHOD FOR MANUFACTURING FLUORINE-CONTAINING POLYMER, AND METHOD FOR MANUFACTURING FLUORINE-CONTAINING MONOMER

(71) Applicant: DAIKIN INDUSTRIES, LTD., Osaka (JP)

(72) Inventors: Daichi Mari, Osaka (JP); Makoto Matsuura, Osaka (JP); Yuki Suzuki, Osaka (JP); Shiomi Sakai, Osaka (JP); Tadashi Ino, Osaka (JP)

(73) Assignee: DAIKIN INDUSTRIES, LTD., Osaka (JP)

(21) Appl. No.: 18/791,930

(22) Filed: Aug. 1, 2024

Related U.S. Application Data
(63) Continuation of application No. PCT/JP2023/003081, filed on Jan. 31, 2023.

(30) Foreign Application Priority Data
Feb. 1, 2022 (JP) 2022-014322

(51) Int. Cl. C07D 317/16; C08F 124/00; C08K 5/08; C08K 5/13

(52) U.S. Cl. CPC C07D 317/16; C08F 124/00; C08K 5/08; C08K 5/13; C08K 2201/014

(57) ABSTRACT

A composition in which a fluorine-containing monomer is stabilized, a method for stabilizing a fluorine-containing monomer, and the like are provided. The composition includes a fluorine-containing monomer (M) and a polycyclic aromatic compound (B). The fluorine-containing monomer (M) includes at least one monomer selected from a compound represented by formula (M1), a compound represented by formula (M2), and a compound represented by formula (M3). The polycyclic aromatic compound (B) includes a compound represented by formula (B1).

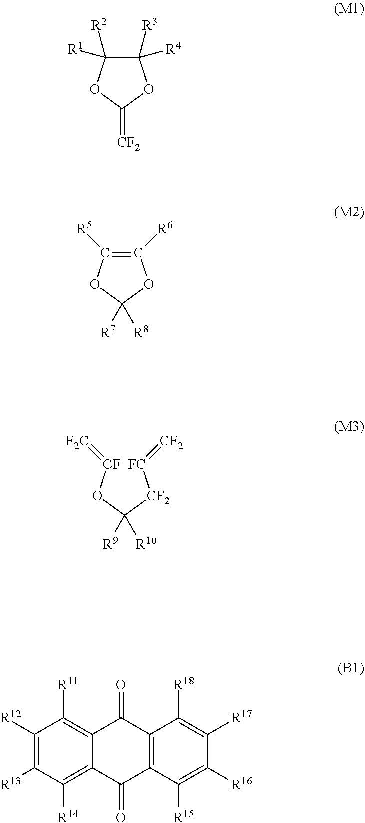

R¹-R¹⁰ each independently represent a fluorine atom, a perfluoroalkyl group, or a perfluoroalkoxy group, and R¹¹-R¹⁸ each independently represent a hydrogen atom or an alkyl group, however, excluding an embodiment in which R¹⁷ represents a tert-butyl group, with R¹¹-R¹⁶ and R¹⁸ being a hydrogen atom.

### TECHNICAL FIELD

The present disclosure relates to a composition containing a fluorine-containing monomer, a method for stabilizing a fluorine-containing monomer, a method for producing a fluorine-containing polymer, a method for producing a fluorine-containing monomer, and the like.

### BACKGROUND ART

Fluorine-containing monomers are used as starting materials for polymerizing fluorine-containing polymers. For example, perfluoro(2-methylene-4-methyl-1,3-dioxolane), which is a fluorine-containing monomer with a ring structure, is polymerized and used in electronic components, optical materials, etc.

Fluorine-containing monomers are prone to decomposition or polymerization during storage. Thus, techniques aimed at stably storing fluorine-containing monomers have been reported (PTL 1 and PTL 2).

### CITATION LIST

### Patent Literature

PTL 1: WO2018/062193A
PTL 2: WO2020/130122A

### SUMMARY

The present disclosure encompasses, for example, the following aspects.

A composition comprising
a fluorine-containing monomer (M), and
a polycyclic aromatic compound (B),
the fluorine-containing monomer (M) being at least one monomer selected from the group consisting of
   a compound represented by formula (M1)

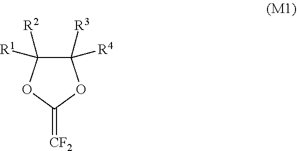

wherein R¹, R², R³, and R⁴ each independently represent a fluorine atom, a perfluoroalkyl group, or a perfluoroalkoxy group,
a compound represented by formula (M2)

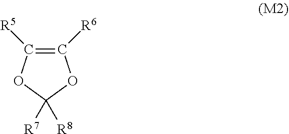

wherein R⁵, R⁶, R⁷, and R⁸ each independently represent a fluorine atom, a perfluoroalkyl group, or a perfluoroalkoxy group, and
a compound represented by formula (M3)

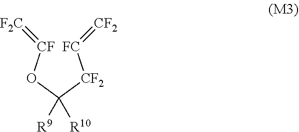

wherein R⁹ and R¹⁰ each independently represent a fluorine atom, a perfluoroalkyl group, or a perfluoroalkoxy group,
   the polycyclic aromatic compound (B) being represented by formula (B1)

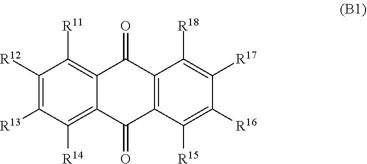

wherein R¹¹, R¹², R¹³, R¹⁴, R¹⁵, R¹⁶, R¹⁷, and R¹⁸ each independently represent a hydrogen atom or an alkyl group, however, excluding an embodiment in which R¹⁷ represents a tert-butyl group, with R¹¹, R¹², R¹³, R¹⁴, R¹⁵, R¹⁶, and R¹⁸ being a hydrogen atom.

### Advantageous Effects

The present disclosure provides a composition containing a fluorine-containing monomer from which the fluorine-containing monomer can be separated according to a method simpler than distillation, a method for stabilizing a fluorine-containing monomer in which the fluorine-containing monomer can be separated according to a method simpler than distillation, and the like. The present disclosure provides a composition containing a fluorine-containing monomer in which the fluorine-containing monomer is more stable even at 0° C., a method for stabilizing a fluorine-containing monomer, and the like. Using the composition containing a fluorine-containing monomer or the method for stabilizing a fluorine-containing monomer according to the present disclosure enables simpler production of a fluorine-containing polymer from a composition in which the fluorine-containing monomer is more stable. Using the composition containing a fluorine-containing monomer or the method for stabilizing a fluorine-containing monomer according to the present disclosure enables simpler production of a fluorine-containing monomer.

### DESCRIPTION OF EMBODIMENTS

The above overview of the present disclosure is not intended to describe each of the disclosed embodiments or all of the implementations of the present disclosure.

The following description of the present disclosure more specifically provides examples of illustrative embodiments.

Guidance is provided through examples in several parts of the present disclosure, and these examples can be used in various combinations.

In each case, the group of examples can function as a non-exclusive and representative group.

All publications, patents, and patent applications cited herein are incorporated herein by reference in their entirety.

### Terms

Unless otherwise specified, the symbols and abbreviations in the present specification can be understood in the context of the present specification in the meanings commonly used in the technical field to which the present disclosure belongs.

In the present specification, the terms “comprise” and “contain” are used with the intention of including the phrases “consisting essentially of” and “consisting of.”

Unless otherwise specified, the steps, treatments, or operations described in the present specification may be performed at room temperature. In the present specification, room temperature can refer to a temperature within the range of 10 to 40° C.

In the present specification, the phrase “Cₙ-Cₘ” (n and m are each a number) indicates that the number of carbon atoms is n or more and m or less, as a person skilled in the art would generally understand.

In the present specification, the description of compounds can include all stereoisomers (enantiomers, diastereomers, geometric isomers, etc.) unless otherwise specified by a person skilled in the art.

In the present specification, unless otherwise specified, examples of “alkyl” include linear, branched, and cyclic alkyl groups. The alkyl can be a linear or branched alkyl group. The number of carbon atoms of the alkyl group can be, for example, 1 to 12, 1 to 6, 1 to 5, 1 to 4, 1 to 3, 6, 5, 4, 3, 2, or 1. Examples of alkyl include linear or branched alkyl groups, such as methyl, ethyl, propyl, isopropyl, butyl, isobutyl, sec-butyl, tert-butyl, pentyl, isopentyl, neopentyl, hexyl, heptyl, octyl, nonyl, and decyl; and cyclic alkyl groups, such as cyclopropyl, cyclobutyl, cyclopentyl, cyclohexyl, cycloheptyl, and cyclooctyl.

In the present specification, unless otherwise specified, “fluoroalkyl” includes linear, branched, or cyclic alkyl groups in which at least one hydrogen atom is replaced by a fluorine atom. “Fluoroalkyl” may be linear or branched alkyl. The number of carbon atoms in “fluoroalkyl” may be, for example, 1 to 12, 1 to 6, 1 to 5, 1 to 4, 1 to 3, 6, 5, 4, 3, 2, or 1.

The number of fluorine atoms in “fluoroalkyl” may be 1 or more (e.g., 1 to 3, 1 to 5, 1 to 9, 1 to 11, or 1 to the maximum substitutable number).

“Fluoroalkyl” includes perfluoroalkyl. “Perfluoroalkyl” is alkyl in which all of the hydrogen atoms are replaced by fluorine atoms.

Examples of perfluoroalkyl include trifluoromethyl (CF₃—), pentafluoroethyl (C₂F₅—), perfluoropropyl (e.g., CF₃CF₂CF₂— and (CF₃)₂CF—), perfluorobutyl (e.g., CF₃CF₂CF₂CF₂—, (CF₃)₂CFCF₂—, CF₃CF CF₃)₂CF₂—, and (CF₃)₃C—), and perfluoropentyl (e.g., CF₃CF₂CF₂CF₂CF₂—, (CF₃)₂CFCF₂CF₂—, CF₃CF₂CF(CF₃)CF₂—, CF₃CF₂CF₂CF(CF₃)—, and CF₃C(CF₃)₂CF₂—).

Specific examples of fluoroalkyl include those listed as examples, such as perfluoroalkyl, monofluoromethyl, difluoromethyl, 2,2,2-trifluoroethyl (CF₃CH₂—), tetrafluoropropyl (e.g., HCF₂CF₂CH₂—), hexafluoropropyl (e.g., (CF₃)₂CH—), and octafluoropentyl (e.g., HCF₂CF₂CF₂CF₂CH₂—).

In the present specification, unless otherwise indicated, “alkoxy” may be a group represented by RO-wherein R represents an alkyl group. “Alkoxy” includes linear, branched, or cyclic alkoxy groups. “Alkoxy” may be linear or branched alkoxy. The number of carbon atoms in “alkoxy” may be, for example, 1 to 12, 1 to 6, 1 to 5, 1 to 4, 1 to 3, 6, 5, 4, 3, 2, or 1.

Examples of alkoxy include linear or branched alkoxy groups, such as methoxy, ethoxy, propoxy, isopropoxy, butoxy, isobutoxy, sec-butoxy, tert-butoxy, pentyloxy, isopentyloxy, neopentyloxy, hexyloxy, heptyloxy, octyloxy, nonyloxy, and decyloxy; and cyclic alkoxy, such as cyclopropyloxy, cyclobutyloxy, cyclopentyloxy, cyclohexyloxy, cycloheptyloxy, and cyclooctyloxy.

In the present specification, unless otherwise specified, “fluoroalkoxy” is alkoxy in which at least one hydrogen atom is replaced by a fluorine atom. “Fluoroalkoxy” may be linear or branched fluoroalkoxy.

The number of carbon atoms in “fluoroalkoxy” may be, for example, 1 to 12, 1 to 6, 1 to 5, 1 to 4, 1 to 3, 6, 5, 4, 3, 2, or 1.

The number of fluorine atoms in “fluoroalkoxy” may be 1 or more (e.g., 1 to 3, 1 to 5, 1 to 9, 1 to 11, or 1 to the maximum substitutable number).

“Fluoroalkoxy” includes perfluoroalkoxy. “Perfluoroalkoxy” is alkoxy in which all of the hydrogen atoms are replaced by fluorine atoms.

Examples of perfluoroalkoxy include trifluoromethyloxy (CF₃O—), pentafluoroethyloxy (C₂F₅O—), perfluoropropyloxy (e.g., CF₃CF₂CF₂O— and (CF₃)₂CFO—) perrfluorobutyloxy (e.g., CF₃CF₂CF₂CF₂O—, (CF₃)₂CFCF₂O—, CF₃CF(CF₃)CF₂O—, and (CF₃)₃CO—), and perfluoopentyloxy (e.g., CF₃CF₂CF₂CF₂CF₂O—, (CF₃)₂CFCF₂CF₂O—, CF₃CF₂CF(CF₃)CF₂O—, CF₃CF₂CF₂CF(CF₃)O—, and CF₃C(CF₃)₂CF₂O—).

Specific examples of fluoroalkoxy include those listed as examples, such as perfluoroalkoxy, monofluoromethoxy, difluoromethoxy, 2,2,2-trifluoroethyloxy (CF₃CH₂O—), tetrafluoropropyloxy (e. g., HCF₂CF₂CH₂O—), hexafluoropropyloxy (e.g., (CF₃)₂CHO—), and octafluoropentyloxy (e.g., HCF₂CF₂CF₂CF₂CH₂O—).

### Composition

One embodiment of the present disclosure is a composition containing a fluorine-containing monomer (M) and a polycyclic aromatic compound (B). This composition limits the decomposition and polymerization of the fluorine-containing monomer (M) and thus stabilizes the fluorine-containing monomer (M). Moreover, the polycyclic aromatic compound (B) can be separated from this composition according to a simple method. Thus, the composition of the present disclosure is advantageous in terms of stable storage of the fluorine-containing monomer (M) and is also useful as a supply source of the fluorine-containing monomer (M). After the fluorine-containing monomer (M) is produced, the polycyclic aromatic compound (B) is mixed with the fluorine-containing monomer (M) to form the composition of the present disclosure, which is then stored. When the fluorine-containing monomer (M) is used (e.g., when the monomer is polymerized to produce a fluorine-containing polymer), the polycyclic aromatic compound (B) is separated and removed from the composition, and the resulting fluorine-containing monomer is polymerized to produce a fluorine-containing polymer (P).

The composition of the present disclosure may contain other components, such as impurities, that are mixed in during the production process of the fluorine-containing monomer (M).

### Fluorine-Containing Monomer (M)

The fluorine-containing monomer (M) is at least one monomer selected from the group consisting of
a compound represented by formula (M1)

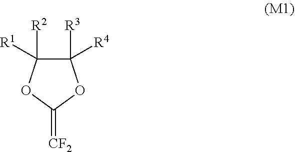

wherein R¹, R², R³, and R⁴ each independently represent a fluorine atom, a perfluoroalkyl group, or a perfluoroalkoxy group (in the present specification, “monomer (M1)”),
a compound represented by formula (M2)

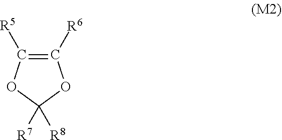

wherein R⁵, R⁶, R⁷, and R⁸ each independently represent a fluorine atom, a perfluoroalkyl group, or a perfluoroalkoxy group (in the present specification, “monomer (M2)”), and
a compound represented by formula (M3)

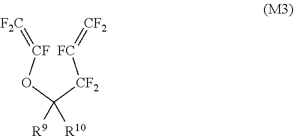

wherein R⁹ and R¹⁰ each independently represent a fluorine atom, a perfluoroalkyl group, or a perfluoroalkoxy group (in the present specification, “monomer (M3)”). The monomer may be of one type or a combination of two or more types.

The production method of the fluorine-containing monomer (M) is known. In the present disclosure, the fluorine-containing monomer (M) may be produced by using a known production method. For example, the monomer (M1) can be produced according to the method described in, for example, JP2005-002014A, WO2020/166632A, or WO2020/230822A.

### Monomer (M1)

R¹, R², R³, and R⁴ may each independently represent a fluorine atom, a C1-C5 perfluoroalkyl group, or a C1-C5 perfluoroalkoxy group.

R¹, R², R³, and R⁴ may each independently represent a fluorine atom, a C1-C4 perfluoroalkyl group, or a C1-C4 perfluoroalkoxy group.

R¹, R², R³, and R⁴ may each independently represent a fluorine atom, a C1-C3 perfluoroalkyl group, or a C1-C3 perfluoroalkoxy group.

R¹, R², R³, and R⁴ may each independently represent a fluorine atom, trifluoromethyl, pentafluoroethyl, trifluoromethyloxy, or pentafluoroethyloxy.

R¹, R², R³, and R⁴ may each independently represent a fluorine atom, trifluoromethyl, pentafluoroethyl, or trifluoromethyloxy.

If at least one group of R¹, R², R³, and R⁴ is a fluorine atom, and the remaining groups are two or more, the remaining groups may be independently a C1-C2 perfluoroalkyl group or a C1-C2 perfluoroalkoxy group.

If at least two groups of R¹, R², R³, and R⁴ are a fluorine atom, and the remaining groups are two, the remaining groups may be independently a C1-C2 perfluoroalkyl group or a C1-C2 perfluoroalkoxy group.

If at least three of R¹, R², R³, and R⁴ are a fluorine atom, the remaining group may be a C1-C2 perfluoroalkyl group or a C1-C2 perfluoroalkoxy group.

If at least three groups of R¹, R², R³, and R⁴ are a fluorine atom, the remaining group may be a C1-C2 perfluoroalkyl group.

R¹, R², R³, and R⁴ may all be a fluorine atom.

The monomer (M1) is preferably a compound represented by the following formula (M1-1) (perfluoro(2-methylene-4-methyl-1,3-dioxolane; also referred to as “monomer (M1-1)” in the present specification) or a compound represented by the following formula (M1-2) (perfluoro(2-methylene-1,3-dioxolane); also referred to as “monomer (M1-2)” in the present specification).

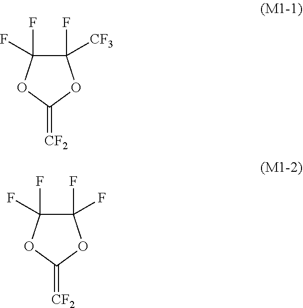

### Monomer (M2)

R⁵, R⁶, R⁷, and R⁸ may each independently represent a fluorine atom, a C1-C5 perfluoroalkyl group, or a C1-C5 perfluoroalkoxy group.

R⁵, R⁶, R⁷, and R⁸ may each independently represent a fluorine atom, a C1-C4 perfluoroalkyl group, or a C1-C4 perfluoroalkoxy group.

R⁵, R⁶, R⁷, and R⁸ may each independently represent a fluorine atom, a C1-C3 perfluoroalkyl group, or a C1-C3 perfluoroalkoxy group.

R⁵, R⁶, R⁷, and R⁸ may each independently represent a fluorine atom, trifluoromethyl, pentafluoroethyl, trifluoromethyloxy, or pentafluoroethyloxy.

R⁵, R⁶, R⁷, and R⁸ may each independently represent a fluorine atom, trifluoromethyl, pentafluoroethyl, or trifluoromethyloxy.

If at least one group of R⁵, R⁶, R⁷, and R⁸ is a fluorine atom, and the remaining groups are two or more, the remaining groups may be independently a C1-C5 perfluoroalkyl group or a C1-C2 perfluoroalkoxy group.

If at least two groups of R⁵, R⁶, R⁷, and R⁸ are a fluorine atom, and the remaining groups are two, the remaining groups may be independently a C1-C5 perfluoroalkyl group or a C1-C2 perfluoroalkoxy group. In this case, it is preferred that R⁵ and R⁶ be both a fluorine atom, and R⁷ and R⁸ be each independently a C1-C2 perfluoroalkyl group or a C1-C2 perfluoroalkoxy group.

If at least three groups of R⁵, R⁶, R⁷, and R⁸ are a fluorine atom, the remaining group may be a C1-C5 perfluoroalkyl group or a C1-C2 perfluoroalkoxy group. In this case, it is preferred that R⁵, R⁷, and R⁸ all be a fluorine atom, and R⁶ be a C1-C5 perfluoroalkyl group or a C1-C2 perfluoroalkoxy group.

If at least three groups of R⁵, R⁶, R⁷, and R⁸ are a fluorine atom, the remaining group may be a Cl-C2 perfluoroalkoxy group. In this case, it is preferred that R⁵, R⁷, and R⁸ all be a fluorine atom, and R⁶ be a C1-C2 perfluoroalkoxy group.

R⁵, R⁶, R⁷, and R⁸ may all be a fluorine atom.

Preferable monomers (M2) include a compound represented by the following formula (M2-1) and a compound represented by the following formula (M2-2) (which are also referred to as “monomer (M2-1)” and “monomer (M2-2)” respectively in the present specification).

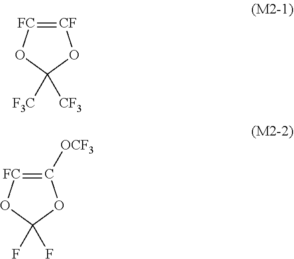

### Monomer (M3)

R⁹ and R¹⁰ may each independently represent a fluorine atom, a C1-C5 perfluoroalkyl group, or a C1-C5 perfluoroalkoxy group.

R⁹ and R¹⁰ may each independently represent a fluorine atom, a C1-C4 perfluoroalkyl group, or a C1-C4 perfluoroalkoxy group.

R⁹ and R¹⁰ may each independently represent a fluorine atom, a C1-C3 perfluoroalkyl group, or a C1-C3 perfluoroalkoxy group.

R⁹ and R¹⁰ may each independently represent a fluorine atom, a C1-C2 perfluoroalkyl group, or a C1-C2 perfluoroalkoxy group.

R⁹ and R¹⁰ may each independently represent a fluorine atom, trifluoromethyl, pentafluoroethyl, or trifluoromethyloxy.

R⁹ and R¹⁰ may both be a fluorine atom.

Preferable monomers (M3) include a compound represented by the following formula (M3-1) (which is also referred to as “monomer (M3-1)” in the present specification).

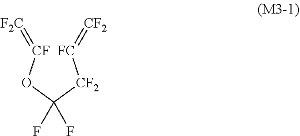

### Polycyclic Aromatic Compound (B)

The polycyclic aromatic compound (B) is at least one compound selected from the group consisting of compounds represented by formula (B1)

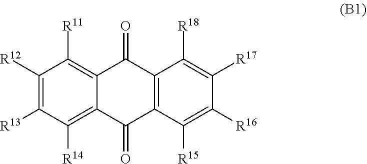

wherein R¹¹, R¹², R¹³, R¹⁴, R¹⁵, R¹⁶, R¹⁷, and R¹⁸ each independently represent a hydrogen atom or an alkyl group, however, excluding the case in which if the present disclosure relates to the composition, R¹⁷ is a tert-butyl group, and R¹¹, R¹², R¹³, R¹⁴, R¹⁵, R¹⁶, and R¹⁸ are a hydrogen atom (which is also referred to as “compound (B1)” in the present specification).

If the present disclosure relates to the composition, the compound (B1) does not encompass a compound in which R¹⁷ is a tert-butyl group, and R¹¹, R¹², R¹³, R¹⁴, R¹⁵, R¹⁶, and R¹⁸ are a hydrogen atom (i.e., 2-(tert-butyl)anthracene-9,10-dione), as stated above. However, when the present disclosure relates to the stabilization method or the production method, the compound (B1) may encompass 2-(tert-butyl)anthracene-9,10-dione.

The polycyclic aromatic compound (B) may be of one type or a combination of two or more types.

The polycyclic aromatic compound (B) can limit the decomposition and polymerization of the fluorine-containing monomer (M). In particular, the polycyclic aromatic compound (B) is highly effective in limiting the decomposition and polymerization of the fluorine-containing monomer (M) even at about 0° C.

Because the polycyclic aromatic compound (B) is poorly soluble in the fluorine-containing monomer (M), the polycyclic aromatic compound (B) can be separated from the fluorine-containing monomer (M) easily or at low cost.

Because the polycyclic aromatic compound (B) is generally poorly dissolved at around room temperature due to its high melting point and has low hygroscopicity, the polycyclic aromatic compound (B) is easy to handle.

### Compound (B1)

When the present disclosure relates to the composition, the embodiment in which R¹⁷ is a tert-butyl group, and R¹¹, R¹², R¹³, R¹⁴, R¹⁵, R¹⁶, and R¹⁸ are a hydrogen atom in formula (B1) is excluded. When the present disclosure relates to the method for stabilizing a fluorine-containing monomer (M), the method for producing a fluorine-containing polymer (P), or the method for producing a fluorine-containing monomer (M), R¹⁷ may be a tert-butyl group, with R¹¹, R¹², R¹³, R¹⁴, R¹⁵, R¹⁶, and R¹⁸ being a hydrogen atom in formula (B1).

R¹¹, R¹², R¹³, R¹⁴, R¹⁵, R¹⁶, R¹⁷, and R¹⁸ may each independently represent a hydrogen atom or a C1-C5 alkyl group.

R¹¹, R¹², R¹³, R¹⁴, R¹⁵, R¹⁶, R¹⁷, and R¹⁸ may each independently represent a hydrogen atom or a C1-C4 alkyl group.

In these cases, when the present disclosure relates to the composition, the embodiment in which R¹⁷ is a tert-butyl group and R¹¹, R¹², R¹³, R¹⁴, R¹⁵, R¹⁶, and R¹⁸ are a hydrogen atom is excluded.

R¹¹, R¹², R¹³, R¹⁴, R¹⁵, R¹⁶, R¹⁷, and R¹⁸ may each independently represent a hydrogen atom or a C1-C3 alkyl group.

R¹¹, R¹², R¹³, R¹⁴, R¹⁵, R¹⁶, R¹⁷, and R¹⁸ may each independently represent a hydrogen atom or a C1-C2 alkyl group.

R¹¹, R¹², R¹³, R¹⁴, R¹⁵, R¹⁶, R¹⁷, and R¹⁸ may each independently represent a hydrogen atom or methyl.

R¹¹, R¹², R¹³, R¹⁴, R¹⁵, R¹⁶, R¹⁷, and R¹⁸ may all be a hydrogen atom.

Five groups of R¹¹, R¹², R¹³, R¹⁴, R¹⁵, R¹⁶, R¹⁷, and R¹⁸ may be a hydrogen atom, with the other three groups being each independently a hydrogen atom or an alkyl group.

Five groups of R¹¹, R¹², R¹³, R¹⁴, R¹⁵, R¹⁶, R¹⁷, and R¹⁸ may be a hydrogen atom, with the other three groups being each independently a hydrogen atom or a C1-C5 alkyl group.

Five groups of R¹¹, R¹², R¹³, R¹⁴, R¹⁵, R¹⁶, R¹⁷, and R¹⁸ may be a hydrogen atom, with the other three groups being each independently a hydrogen atom or a C1-C4 alkyl group.

Five groups of R¹¹, R¹², R¹³, R¹⁴, R¹⁵, R¹⁶, R¹⁷, and R¹⁸ may be a hydrogen atom, with the other three groups being each independently a hydrogen atom or a C1-C3 alkyl group.

Five groups of R¹¹, R¹², R¹³, R¹⁴, R¹⁵, R¹⁶, R¹⁷, and R¹⁸ may be a hydrogen atom, with the other three groups being each independently a hydrogen atom or a C1-C2 alkyl group.

Five groups of R¹¹, R¹², R¹³, R¹⁴, R¹⁵, R¹⁶, R¹⁷, and R¹⁸ may be a hydrogen atom, with the other three groups being each independently a hydrogen atom or methyl.

Six groups of R¹¹, R¹², R¹³, R¹⁴, R¹⁵, R¹⁶, R¹⁷, and R¹⁸ may be a hydrogen atom, with the other two groups being each independently a hydrogen atom or an alkyl group.

Six groups of R¹¹, R¹², R¹³, R¹⁴, R¹⁵, R¹⁶, R¹⁷, and R¹⁸ may be a hydrogen atom, with the other two groups being each independently a hydrogen atom or a C1-C5 alkyl group.

Six groups of R¹¹, R¹², R¹³, R¹⁴, R¹⁵, R¹⁶, R¹⁷, and R¹⁸ may be a hydrogen atom, with the other two groups being each independently a hydrogen atom or a C1-C4 alkyl group.

Six groups of R¹¹, R¹², R¹³, R¹⁴, R¹⁵, R¹⁶, R¹⁷, and R¹⁸ may be a hydrogen atom, with the other two groups being each independently a hydrogen atom or a C1-C3 alkyl group.

Six groups of R¹¹, R¹², R¹³, R¹⁴, R¹⁵, R¹⁶, R¹⁷, and R¹⁸ may be a hydrogen atom, with the other two groups being each independently a hydrogen atom or a C1-C2 alkyl group.

Six groups of R¹¹, R¹², R¹³, R¹⁴, R¹⁵, R¹⁶, R¹⁷, and R¹⁸ may be a hydrogen atom, with the other two groups being each independently a hydrogen atom or methyl.

Seven groups of R¹¹, R¹², R¹³, R¹⁴, R¹⁵, R¹⁶, R¹⁷, and R¹⁸ may be a hydrogen atom, with the remaining group being a hydrogen atom or an alkyl group.

Seven groups of R¹¹, R¹², R¹³, R¹⁴, R¹⁵, R¹⁶, R¹⁷, and R¹⁸ may be a hydrogen atom, with the remaining group being a hydrogen atom or a C1-C5 alkyl group.

Seven groups of R¹¹, R¹², R¹³, R¹⁴, R¹⁵, R¹⁶, R¹⁷, and R¹⁸ may be a hydrogen atom, with the remaining group being a hydrogen atom or a C1-C4 alkyl group.

In these cases, when the present disclosure relates to the composition, the embodiment in which R¹⁷ is a tert-butyl group, and R¹¹, R¹², R¹³, R¹⁴, R¹⁵, R¹⁶, and R¹⁸ are a hydrogen atom is excluded.

Seven groups of R¹¹, R¹², R¹³, R¹⁴, R¹⁵, R¹⁶, R¹⁷, and R¹⁸ may be a hydrogen atom, with the remaining group being a hydrogen atom or a C1-C3 alkyl group.

Seven groups of R¹¹, R¹², R¹³, R¹⁴, R¹⁵, R¹⁶, R¹⁷, and R¹⁸ may be a hydrogen atom, with the remaining group being a hydrogen atom or a C1-C2 alkyl group.

Seven groups of R¹¹, R¹², R¹³, R¹⁴, R¹⁵, R¹⁶, R¹⁷, and R¹⁸ may be a hydrogen atom, with the remaining group being a hydrogen atom or methyl.

The number of alkyl groups in the compound (B1) may be 0, 1, 2, 3, 4, 5, 6, 7, or 8, preferably 0, 1, 2, 3, or 4, more preferably 0, 1, 2, or 3, even more preferably 0, 1, or 2, and particularly preferably 0 or 1.

When the compound (B1) has an alkyl group, at least one of R¹², R¹³, R¹⁶, and R¹⁷ may be an alkyl group. In this case, R¹¹, R¹⁴, R¹⁵, and R¹⁸ are independently a hydrogen atom or an alkyl group, and all preferably are a hydrogen atom.

Preferable compounds (B1) include the compounds represented by the following formulas (B1-1) to (B1-7). The compounds represented by the following formulas (B1-1) to (B1-7) are also referred to as “compound (B1-1),” “compound (B1-2),” “compound (B1-3),” “compound (B1-4),” “compound (B1-5),” “compound (B1-6),” and “compound (B1-7),” respectively.

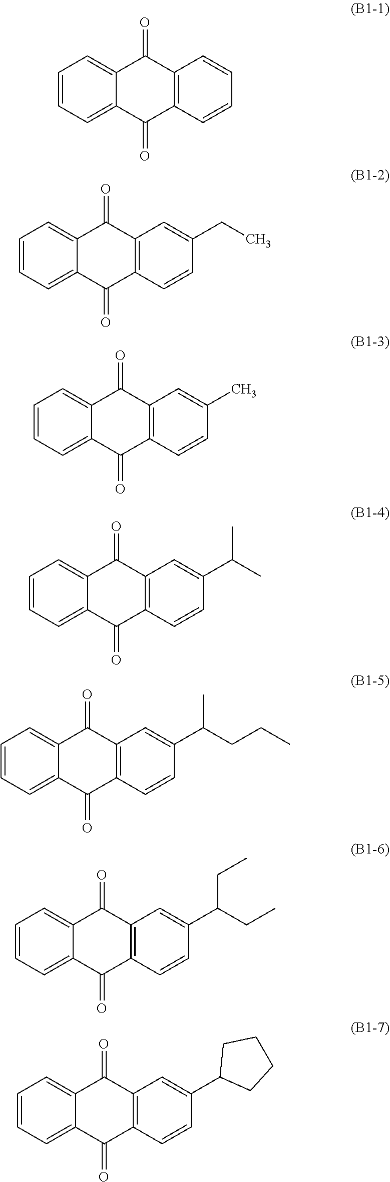

In the composition of the present disclosure, the content of the fluorine-containing monomer (M) based on the mass of the composition may be, for example, 10 to 99.999 mass %, 90 to 99.999 mass %, 92 to 99.999 mass %, 94 to 99.999 mass %, 95 to 99.999 mass %, 96 to 99.999 mass %, 97 to 99.999 mass %, 90 to 99.99 mass %, 92 to 99.99 mass %, 94 to 99.99 mass %, 95 to 99.99 mass %, 96 to 99.99 mass %, 97 to 99.99 mass %, 90 to 99.9 mass %, 92 to 99.9 mass8, 94 to 99.9 mass8, 95 to 99.9 mass %, 96 to 99.9 mass %, 97 to 99.9 mass %, 90 to 99 mass %, 92 to 99 mass %, 94 to 99 mass %, 95 to 99 mass %, 96 to 99 mass %, or 97 to 99 mass %, preferably 50 to 99.999 mass %, and more preferably 90 to 99.999 mass %.

In the composition of the present disclosure, the content of the polycyclic aromatic compound (B) based on the mass of the composition may be, for example, 0.001 to 90 mass %, 0.001 to 10 mass %, 0.001 to 8 mass %, 0.001 to 6 mass %, 0.001 to 5 mass %, 0.001 to 4 mass %, 0.001 to 3 mass %, 0.01 to 10 mass %, 0.01 to 8 mass %, 0.01 to 6 mass %, 0.01 to 5 mass %, 0.01 to 4 mass %, 0.01 to 3 mass %, 0.1 to 10 mass %, 0.1 to 8 mass %, 0.1 to 6 mass %, 0.1 to 5 mass %, 0.1 to 4 mass %, 0.1 to 3 mass %, 1 to 10 mass %, 1 to 8 mass %, 1 to 6 mass %, 1 to 5 mass %, 1 to 4 mass %, or 1 to 3 mass %, preferably 0.001 to 50 mass %, and more preferably 0.001 to 10 mass %.

The composition of the present disclosure may contain other components in addition to the fluorine-containing monomer (M) and the polycyclic aromatic compound (B). The content of the other components may be, for example, 0.001 to 8 mass %, 0.001 to 6 mass %, or 0.001 to 5 mass %, based on the mass of the composition.

Examples of other components include impurities mixed in during the production process of the fluorine-containing monomer (M), phenol compounds optionally substituted with an alkyl group and/or an alkoxy group, and naphthoquinone compounds optionally substituted with an alkyl group.

The composition of the present disclosure may be a composition obtained by mixing a product liquid of the fluorine-containing monomer (M) generated during the production of the fluorine-containing monomer (M) with the polycyclic aromatic compound (B), or may be a composition obtained by purifying the product liquid of the fluorine-containing monomer (M) and then mixing it with the polycyclic aromatic compound (B).

The impurities include, for example, the compounds represented by the following formulas: In the following formulas, R⁵³, R⁵⁴, R⁵⁵, and R⁵⁶ each independently represent a fluorine atom, a perfluoroalkyl group, or a perfluoroalkoxy group. For R⁵³, R⁵⁴, R⁵⁵, and R⁵⁶, the above descriptions regarding R¹, R², R³, and R⁴ are respectively applied.

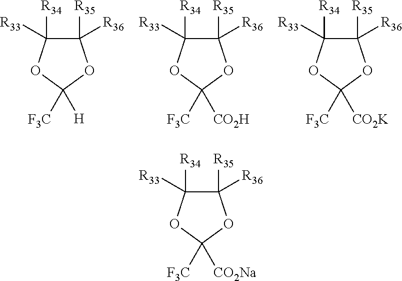

Examples of the phenol compounds optionally substituted with an alkyl group and/or an alkoxy group include a phenol compound (C) represented by formula (C1):

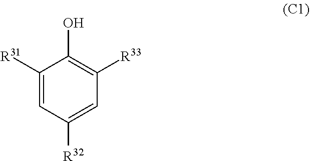

wherein R³¹, R³², and R³³ each independently represent a hydrogen atom, a C1-C5 alkyl group, or a C1-C5 alkoxy group.

R³¹, R³², and R³³ may each independently represent a hydrogen atom, a C1-C4 alkyl group, or a C1-C4 alkoxy group.

R³¹, R³², and R³³ may each independently represent a hydrogen atom, a C1-C3 alkyl group, or a C1-C3 alkoxy group.

R³¹, R³², and R³³ may each independently represent a hydrogen atom, a C1-C2 alkyl group, or a C1-C2 alkoxy group.

R³¹, R³², and R³³ may each independently represent a hydrogen atom, methyl, or methoxy.

Preferable phenol compounds (C) include the compound represented by the following formula (C1-1) (which is also referred to as “compound (C1-1)” in the present specification), a compound represented by the following formula (C1-2) (which is also referred to as “compound (C1-2)” in the present specification), and a compound represented by the following formula (C1-3) (which is also referred to as “compound (C1-3)” in the present specification).

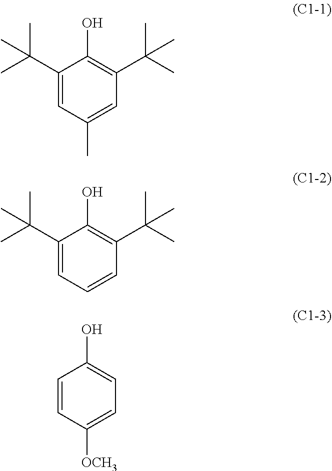

Examples of the naphthoquinone compounds optionally substituted with an alkyl group include a naphthoquinone compound (D) represented by the following formula (D1):

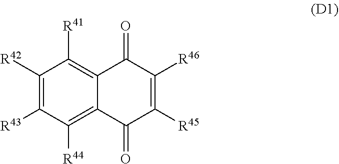

wherein R⁴¹, R⁴², R⁴³, R⁴⁴, R⁴⁵, and R⁴⁶ each independently represent a hydrogen atom or a C1-C5 alkyl group.

R⁴¹, R⁴², R⁴³, R⁴⁴, R⁴⁵, and R⁴⁶ may each independently represent a hydrogen atom or a C1-C5 alkyl group.

R⁴¹, R⁴², R⁴³, R⁴⁴, R⁴⁵, and R⁴⁶ may each independently represent a hydrogen atom or a C1-C4 alkyl group.

R⁴¹, R⁴², R⁴³, R⁴⁴, R⁴⁵, and R⁴⁶ may each independently represent a hydrogen atom or a C1-C3 alkyl group.

R⁴¹, R⁴², R⁴³, R⁴⁴, R⁴⁵, and R⁴⁶ may each independently represent a hydrogen atom or a C1-C2 alkyl group.

R⁴¹, R⁴², R⁴³, R⁴⁴, R⁴⁵, and R⁴⁶ may each independently represent a hydrogen atom or methyl.

R⁴¹, R⁴², R⁴³, R⁴⁴, R⁴⁵, and R⁴⁶ may all represent a hydrogen atom.

Three groups of R⁴¹, R⁴², R⁴³, R⁴⁴, R⁴⁵, and R⁴⁶ may be a hydrogen atom, with the other three groups being each independently a hydrogen atom or a C1-C5 alkyl group.

Three groups of R⁴¹, R⁴², R⁴³, R⁴⁴, R⁴⁵, and R⁴⁶ may be a hydrogen atom, with the other three groups being each independently a hydrogen atom or a C1-C4 alkyl group.

Three groups of R⁴¹, R⁴², R⁴³, R⁴⁴, R⁴⁵, and R⁴⁶ may be a hydrogen atom, with the other three groups being each independently a hydrogen atom or a C1-C3 alkyl group.

Three groups of R⁴¹, R⁴², R⁴³, R⁴⁴, R⁴⁵, and R⁴⁶ may be a hydrogen atom, with the other three groups being each independently a hydrogen atom or a C1-C2 alkyl group.

Three groups of R⁴¹, R⁴², R⁴³, R⁴⁴, R⁴⁵, and R⁴⁶ may be a hydrogen atom, with the other three groups being each independently a hydrogen atom or methyl.

Four groups of R⁴¹, R⁴², R⁴³, R⁴⁴, R⁴⁵, and R⁴⁶ may be a hydrogen atom, with the other two groups being each independently a hydrogen atom or a C1-C5 alkyl group.

Four groups of R⁴¹, R⁴², R⁴³, R⁴⁴, R⁴⁵, and R⁴⁶ may be a hydrogen atom, with the other two groups being each independently a hydrogen atom or a C1-C4 alkyl group.

Four groups of R⁴¹, R⁴², R⁴³, R⁴⁴, R⁴⁵, and R⁴⁶ may be a hydrogen atom, with the other two groups being each independently a hydrogen atom or a C1-C3 alkyl group.

Four groups of R⁴¹, R⁴², R⁴³, R⁴⁴, R⁴⁵, and R⁴⁶ may be a hydrogen atom, with the other two groups being each independently a hydrogen atom or a C1-C2 alkyl group.

Four groups of R⁴¹, R⁴², R⁴³, R⁴⁴, R⁴⁵, and R⁴⁶ may be a hydrogen atom, with the other two groups being each independently a hydrogen atom or methyl.

Five groups of R⁴¹, R⁴², R⁴³, R⁴⁴, R⁴⁵, and R⁴⁶ may be a hydrogen atom, with the other group being a hydrogen atom or a C1-C5 alkyl group.

Five groups of R⁴¹, R⁴², R⁴³, R⁴⁴, R⁴⁵, and R⁴⁶ may be a hydrogen atom, with the other group being a hydrogen atom or a C1-C4 alkyl group.

Five groups of R⁴¹, R⁴², R⁴³, R⁴⁴, R⁴⁵, and R⁴⁶ may be a hydrogen atom, with the other group being a hydrogen atom or a C1-C3 alkyl group.

Five groups of R⁴¹, R⁴², R⁴³, R⁴⁴, R⁴⁵, and R⁴⁶ may be a hydrogen atom, with the other group being a hydrogen atom or a C1-C2 alkyl group.

Five groups of R⁴¹, R⁴², R⁴³, R⁴⁴, R⁴⁵, and R⁴⁶ may be a hydrogen atom, with the other group being a hydrogen atom or methyl.

The number of C1-C5 alkyl groups in the naphthoquinone compound (D) may be 0, 1, 2, 3, 4, or 5, preferably 0, 1, 2, 3, or 4, more preferably 0, 1, 2, or 3, even more preferably 0, 1, or 2, and particularly preferably 0 or 1.

If the naphthoquinone compound (D) has a C1-C5 alkyl group, at least one of R⁴⁵ and R⁴⁶ may be a C1-C5 alkyl group. In this case, R⁴¹, R⁴², R⁴³, and R⁴⁴ are independently a hydrogen atom or a C1-C5 alkyl group, and preferably all are a hydrogen atom.

The naphthoquinone compound (D) includes the compounds represented by the following formulas (D1-1) to (D1-9). The compounds represented by the following formulas (D1-1) to (D1-9) are also referred to as “compound (D1-1),” “compound (D1-2),” “compound (D1-3),” “compound (D1-4),” “compound (D1-5),” “compound (D1-6),” “compound (D1-7),” “compound (D1-8),” and “compound (D1-9),” respectively, in the present specification. The naphthoquinone compound (D) is preferably the compound (D1-1) or the compound (D1-2), and more preferably the compound (D1-1).

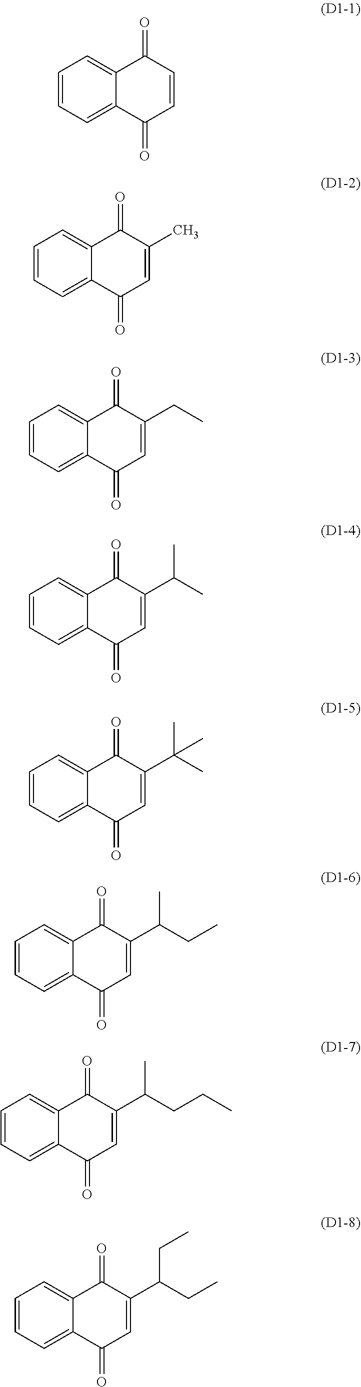

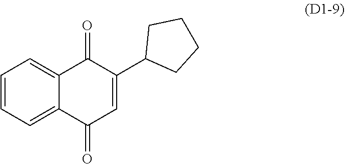 [SMILES: O=C1C=C(C2CCCC2)C(=O)c2ccccc21]

One embodiment of the present disclosure encompasses a composition containing the fluorine-containing monomer (M), the polycyclic aromatic compound (B), and at least one compound selected from the group consisting of the phenol compound (C) and the naphthoquinone compound (D).

One embodiment of the present disclosure encompasses a composition containing the fluorine-containing monomer (M), the polycyclic aromatic compound (B), and at least one compound selected from the group consisting of the compound (C1-1), the compound (C1-2), the compound (C1-3), the compound (D1-1), the compound (D1-2), the compound (D1-3), the compound (D1-4), the compound (D1-5), the compound (D1-6), the compound (D1-7), the compound (D1-8), and the compound (D1-9).

One embodiment of the present disclosure encompasses a composition containing the fluorine-containing monomer (M), the polycyclic aromatic compound (B), and at least one compound selected from the group consisting of the compound (C1-1), the compound (C1-2), the compound (C1-3), the compound (D1-1), and the compound (D1-2).

One embodiment of the present disclosure encompasses the fluorine-containing monomer (M), the polycyclic aromatic compound (B), and the compound (C1-1).

One embodiment of the present disclosure encompasses a composition containing the fluorine-containing monomer (M), the polycyclic aromatic compound (B), and the phenol compound (C).

One embodiment of the present disclosure encompasses a composition containing the fluorine-containing monomer (M), the polycyclic aromatic compound (B), and at least one phenol compound (C) selected from the group consisting of the compound (C1-1), the compound (C1-2), and the compound (C1-3).

One embodiment of the present disclosure encompasses a composition containing the fluorine-containing monomer (M), the polycyclic aromatic compound (B), and the compound (C1-1).

One embodiment of the present disclosure encompasses a composition containing the fluorine-containing monomer (M), the polycyclic aromatic compound (B), and the naphthoquinone compound (D).

One embodiment of the present disclosure encompasses a composition containing the fluorine-containing monomer (M), the polycyclic aromatic compound (B), and at least one naphthoquinone compound (D) selected from the group consisting of the compound (D1-1), the compound (D1-2), the compound (D1-3), the compound (D1-4), the compound (D1-5), the compound (D1-6), the compound (D1-7), the compound (D1-8), and the compound (D1-9).

One embodiment of the present disclosure encompasses a composition containing the fluorine-containing monomer (M), the polycyclic aromatic compound (B), and at least one naphthoquinone compound selected from the group consisting of the compound (D1-1) and the compound (D1-2).

The composition of the present disclosure can be produced by mixing the components to constitute the composition according to an appropriate method. The composition of the present disclosure may also be produced by mixing a product liquid of the fluorine-containing monomer (M) generated during the production of the fluorine-containing monomer (M) with other components. Thus, the product liquid of the fluorine-containing monomer (M) generated during the production of the fluorine-containing monomer (M) can be used as a supply source of the fluorine-containing monomer (M) in the present disclosure.

### Method for Stabilizing Fluorine-containing Monomer (M)

One embodiment of the present disclosure relates to a method for stabilizing a fluorine-containing monomer (M) by mixing the fluorine-containing monomer (M) with a polycyclic aromatic compound (B) and keeping the temperature of the obtained mixture at 10° C. or lower. In this stabilization method, it is important that the fluorine-containing monomer (M) and the polycyclic aromatic compound (B) are present together, and that the mixture of the fluorine-containing monomer (M) and the polycyclic aromatic compound (B) is kept at 10° C. or lower. This is because allowing the polycyclic aromatic compound (B) to be present with the fluorine-containing monomer (M), which is prone to decomposition and polymerization, limits the decomposition and polymerization of the fluorine-containing monomer (M). In this stabilization method, the mixture may contain, in addition to the fluorine-containing monomer (M) and the polycyclic aromatic compound (B), other components, such as impurities mixed in during the production process.

In the stabilization method, the mixture of the fluorine-containing monomer (M) and the polycyclic aromatic compound (B) may be the composition of the present disclosure described above. In the stabilization method, the fluorine-containing monomer (M) and the polycyclic aromatic compound (B) are the same as the fluorine-containing monomer (M) and the polycyclic aromatic compound (B) for the composition described above. In the stabilization method, the other components described above (e.g., the phenol compound (C) and the naphthoquinone compound (D)) may also be added in addition to the fluorine-containing monomer (M) and the polycyclic aromatic compound (B). Thus, the above descriptions regarding the fluorine-containing monomer (M), the polycyclic aromatic compound (B), and other components for the composition are applicable to the stabilization method unless otherwise specified. However, unless otherwise specified, the polycyclic aromatic compound (B) in the stabilization method includes a compound in which R¹⁷ is a tert-butyl group, and R¹¹, R¹², R¹³, R¹⁴, R¹⁵, R¹⁶, and R¹⁸ are a hydrogen atom in formula (B1) (i.e., 2-(tert-butyl)anthracene-9,10-dione). In the stabilization method, the polycyclic aromatic compound (B) may exclude the compound in which R¹⁷ is a tert-butyl group, and R¹¹, R¹², R¹³, R¹⁴, R¹⁵, R¹⁶, and R¹⁸ are a hydrogen atom in formula (B1) (2-(tert-butyl)anthracene-9,10-dione).

In the stabilization method, the proportion of the polycyclic aromatic compound (B) to be mixed may be, for example, 0.001 to 12 mass, 0.001 to 7 mass %, 0.001 to 6 mass %, 0.001 to 5 mass %, 0.001 to 4 mass %, 0.001 to 3 mass %, 0.01 to 12 mass %, 0.01 to 7 mass %, 0.01 to 6 mass, 0.01 to 5 mass %, 0.01 to 4 mass %, 0.01 to 3 mass %, 0.1 to 12 mass %, 0.1 to 7 mass %, 0.1 to 6 mass %, 0.1 to 5 mass, 0.1 to 4 mass %, or 0.1 to 3 mass %, based on the mass of the fluorine-containing monomer (M) taken as 100.

In the stabilization method, as described above, it suffices if the fluorine-containing monomer (M) and the polycyclic aromatic compound (B) are both present. Thus, there is no particular limitation in the method for mixing the fluorine-containing monomer (M) and the polycyclic aromatic compound (B) in the stabilization method. For example, the polycyclic aromatic compound (B) can be added to the fluorine-containing monomer (M), and these are mixed optionally by treatment such as stirring, shaking, or ultrasonic treatment.

Keeping the mixture of the fluorine-containing monomer (M) and the polycyclic aromatic compound (B) at 10° C. or lower limits the decomposition and polymerization of the fluorine-containing monomer (M). This temperature may be 10° C. or lower, preferably 0° C. or lower, and more preferably −10° C. or lower. The lower limit of the temperature is not limited as long as the decomposition and polymerization of the fluorine-containing monomer (M) can be suppressed, and the temperature may be, for example, −80° C. or higher, or −60° C. or higher. These upper and lower temperature limits can be appropriately combined to form a temperature range. The temperature may be −80 to 10° C., −80 to 0° C., −80 to −10° C., −60 to 10° C., −60 to 0° C., or −60 to −10° C.

The mixture of the fluorine-containing monomer (M) and the polycyclic aromatic compound (B) can be stored in a storage container. Examples of storage containers include containers made of resins, such as polyethylene, polypropylene, and polytetrafluoroethylene; containers made of metals, such as stainless steel; and composite containers made of resin and metal. The gas phase to fill the storage container is preferably, but is not limited to, an inert gas, such as nitrogen, argon, or carbon dioxide.

### Method for Producing Fluorine-Containing Polymer (P)

One embodiment of the present disclosure relates to a method for producing a fluorine-containing polymer (P) by polymerizing a fluorine-containing monomer (M), including the mixing step of mixing the fluorine-containing monomer (M) with a polycyclic aromatic compound (B). The structural unit constituting the produced fluorine-containing polymer (P) would be understood from the starting material monomers by those skilled in the art. For example, the structural unit of the fluorine-containing polymer formed by polymerizing the monomer (M1-1), the monomer (M1-2), the monomer (M2-1), the monomer (M2-2), or the monomer (M3-1) is respectively a structural unit represented by the following formula (U1-1) (which is also referred to as “unit (U1-1)” in the present specification), a structural unit represented by the following formula (U1-2) (which is also referred to as “unit (U1-2)” in the present specification), a structural unit represented by formula (U2-1) (which is also referred to as “unit (U2-1)” in the present specification), a structural unit represented by (U2-2) (which is also referred to as “unit (U2-2)” in the present specification), or a structural unit represented by (U3-1) (which is also referred to as “unit (U3-1)” in the present specification).

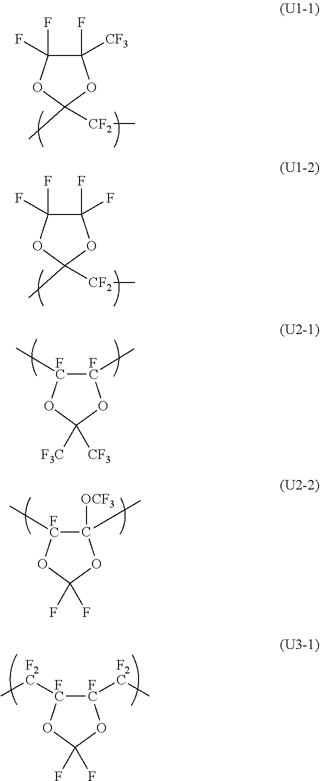

The fluorine-containing polymer (P) may contain the structural unit derived from the fluorine-containing monomer (M) in an amount of, for example, 50 mol % or higher, 60 mol % or higher, 70 mol % or higher, 80 mol % or higher, 90 mol % or higher, or 100% of all of the structural units constituting the fluorine-containing polymer (P).

In the method for producing the fluorine-containing polymer (P) of the present disclosure, the mixture obtained in the mixing step may be the composition of the present disclosure described above. In the production method, the fluorine-containing monomer (M) and the polycyclic aromatic compound (B) are the same as the fluorine-containing monomer (M) and the polycyclic aromatic compound (B) in the composition. However, unless otherwise specified, the polycyclic aromatic compound (B) in the production method also includes a compound in which R¹⁷ is a tert-butyl group, and R¹¹, R¹², R¹³, R¹⁴, R¹⁵, R¹⁶, and R¹⁸ are a hydrogen atom in the formula (B1) (i.e., 2-(tert-butyl)anthracene-9,10-dione). In the production method, the polycyclic aromatic compound (B) may exclude a compound in which R¹⁷ is a tert-butyl group, and R¹¹, R¹², R¹³, R¹⁴, R¹⁵, R¹⁶, and R¹⁸ are a hydrogen atom in formula (B1) (i.e., 2-(tert-butyl)anthracene-9,10-dione). In the production method, the mixture may contain, in addition to the fluorine-containing monomer (M) and the polycyclic aromatic compound (B), the other components (e.g., the phenol compound (C) and the naphthoquinone compound (D)). Thus, the above descriptions regarding the fluorine-containing monomer (M), the polycyclic aromatic compound (B), and other components for the composition and stabilization method are applicable to the method for producing a fluorine-containing polymer (P) unless otherwise specified.

In the mixing step of the method for producing a fluorine-containing polymer (P) of the present disclosure, the proportion of the polycyclic aromatic compound (B) to be mixed may be, for example, 0.001 to 12 mass %, 0.001 to 7 mass %, 0.001 to 6 mass %, 0.001 to 5 mass %, 0.001 to 4 mass %, 0.001 to 3 mass %, 0.01 to 12 mass %, 0.01 to 7 mass %, 0.01 to 6 mass %, 0.01 to 5 mass %, 0.01 to 4 mass %, 0.01 to 3 mass %, 0.1 to 12 mass %, 0.1 to 7 mass %, 0.1 to 6 mass %, 0.1 to 5 mass %, 0.1 to 4 mass %, or 0.1 to 3 mass, based on the mass of the fluorine-containing monomer (M) taken as 100.

The method for producing a fluorine-containing polymer (P) of the present disclosure may further include the separation step of separating the polycyclic aromatic compound (B) from a mixture containing the fluorine-containing monomer (M) and the polycyclic aromatic compound (B) obtained in the mixing step.

The method for separating the polycyclic aromatic compound (B) is not particularly limited as long as separation can be achieved, and any known applicable method can be used. For example, the polycyclic aromatic compound (B) can be separated by adsorption removal using an adsorbent (e.g., silica gel, activated alumina, and zeolite), distillation, filtration, centrifugation, etc.

In the step of separating the polycyclic aromatic compound (B), adsorption removal using an adsorbent (e.g., silica gel, activated alumina, and zeolite) is preferred due to its simplicity in separation compared with distillation.

In the method for producing a fluorine-containing polymer (P) of the present disclosure, the mixture obtained in the mixing step may be mixed with components necessary for polymerization (e.g., a polymerization initiator) and then subjected to polymerization conditions (polymerization step). In this stage, the mixture obtained in the mixing step may be replaced by the fluorine-containing monomer (M) obtained in the separation step described above. Methods for producing the fluorine-containing polymer (P) by polymerizing the fluorine-containing monomer (M) are known, and an example is the production method described in WO2013/018730A. In the production method of the present disclosure, the fluorine-containing polymer (P) can be produced according to a known method as is or with appropriate modifications.

In the method for producing a fluorine-containing polymer (P) of the present disclosure, other monomers, in addition to the fluorine-containing monomer (M), may be used as starting material monomers in polymerization. The type and amount of the other monomers for use may be appropriately adjusted so as to form a desired fluorine-containing polymer (P). The other monomers may be those that form a fluoroolefin unit upon polymerization (e.g., fluoroolefin). The proportion of the fluoroolefin unit may be 50 mol % or lower, preferably 30 mol % or lower, more preferably 20 mol % or lower, even more preferably 10 mol % or lower, and particularly preferably 0 mol % of all of the structural units constituting the fluorine-containing polymer (P).

The fluoroolefin unit is a structural unit formed after polymerization of a monomer containing one or more fluorine atoms and one or more carbon-carbon double bonds.

The atoms constituting the fluoroolefin unit may be only fluorine atoms, halogen atoms other than fluorine atoms, carbon atoms, hydrogen atoms, and oxygen atoms.

The atoms constituting the fluoroolefin unit may be only fluorine atoms, halogen atoms other than fluorine atoms, carbon atoms, and hydrogen atoms.

The atoms constituting the fluoroolefin unit may be only fluorine atoms, carbon atoms, and hydrogen atoms.

The atoms constituting the fluoroolefin unit may be only fluorine atoms and carbon atoms.

The fluoroolefin unit includes at least one unit selected from the group consisting of a fluorine-containing perhaloolefin unit, a vinylidene fluoride unit (—CH₂—CF₂—), a trifluoroethylene unit (—CFH—CF₂—), a pentafluoropropylene unit (—CFH—CF(CF₃)—, —CF₂CF(CHF₂)—), and a 1,1,1,2-tetrafluoro-2-propylene unit (—CH₂—CF(CF₃)—).

The fluorine-containing perhaloolefin unit is a structural unit formed after polymerization of a monomer that contains one or more fluorine atoms and one or more carbon-carbon double bonds with one or more optional halogen atoms other than fluorine atoms.

The fluorine-containing perhaloolefin unit contains at least one selected from the group consisting of a chlorotrifluoroethylene unit (—CFCl—CF₂—), a tetrafluoroethylene unit (—CF₂—CF₂—), a hexafluoropropylene unit (—CF₂—CF(CF₃)—), a perfluoro(methyl vinyl ether) unit (—CF₂—CF(OCF₃)—), a perfluoro(ethyl vinyl ether) unit (—CF₂—CF(OC₂F₅)—), a perfluoro(propyl vinyl ether) unit (—CF₂—CF(OCF₂C₂F₅)—), a perfluoro(butyl vinyl ether) unit (—CF₂—CF(O(CF₂)₂C₂F₅)—), and a perfluoro(2,2-dimethyl-1,3-dioxol) unit (—CF—CAF— wherein A represents a perfluorodioxolane ring formed together with an adjacent carbon atom in the formula with two trifluoromethyl groups bound to the carbon atom at the 2-position of the dioxolane ring).

The fluoroolefin unit may contain at least one selected from the group consisting of a chlorotrifluoroethylene unit, a tetrafluoroethylene unit, a hexafluoropropylene unit, a perfluoro(methyl vinyl ether) unit, and a perfluoro(propyl vinyl ether) unit.

The fluorine-containing polymer (P) may or may not contain one or more other structural units in addition to the structural unit derived from the fluorine-containing monomer (M) and the fluoroolefin unit. Such other structural units include a CH₂═CHRf unit (Rf represents a C1-C10 fluoroalkyl group). Rf may be a C1-C10 perfluoroalkyl group, a C1-C5 fluoroalkyl group, a C1-C5 perfluoroalkyl group, a C1-C3 fluoroalkyl group, or a C1-C3 perfluoroalkyl group.

The proportion of the other structural units may be, for example, 0 mol % or higher and 20 mol % or lower, or 0 mol % or higher and 10 mol % or lower, of all of the structural units constituting the fluorine-containing polymer (P).

The mass average molecular weight of the fluorine-containing polymer (P) is, for example, 10,000 or more and 1,000,000 or less, preferably 30,000 or more and 500,000 or less, and more preferably 50,000 or more and 300,000 or less. A mass average molecular weight within these ranges is advantageous in terms of durability.

Those skilled in the art would understand the monomers corresponding to the structural units constituting the fluorine-containing polymer (P). For example, monomers corresponding to a tetrafluoroethylene unit, a hexafluoropropylene unit, and a vinylidene fluoride unit are respectively tetrafluoroethylene (CF₂═CF₂), hexafluoropropylene (CF₃CF═CF₂), and vinylidene fluoride (CH₂═CF₂).

The fluoroolefin (monomer) corresponding to a fluoroolefin unit may be, for example, at least one selected from the group consisting of fluorine-containing perhaloolefin, vinylidene fluoride, trifluoroethylene, pentafluoropropylene, and 1,1,1,2-tetrafluoro-2-propylene. The fluoroolefin may preferably be at least one selected from the group consisting of chlorotrifluoroethylene, tetrafluoroethylene, hexafluoropropylene, perfluoro(methyl vinyl ether), and perfluoro(propyl vinyl ether).

The fluorine-containing perhaloolefin may be at least one selected from the group consisting of chlorotrifluoroethylene, tetrafluoroethylene, hexafluoropropylene, perfluoro(methyl vinyl ether), perfluoro(ethyl vinyl ether), perfluoro(propyl vinyl ether), perfluoro(butyl vinyl ether), and perfluoro(2,2-dimethyl-1,3-dioxole).

As described above, the polymerization step can be performed according to a known method or a known method with some modifications. For example, polymerization can be performed by adding a solvent, a polymerization initiator, etc. to the mixture obtained in the mixing step or the monomer obtained in the separation step, as necessary. The polymerization step is, for example, a method that includes dissolving or dispersing an appropriate amount of a starting material monomer in a solvent (e.g., an aprotic solvent) as necessary, adding a polymerization initiator as necessary, and performing polymerization (e.g., radical polymerization, bulk polymerization, solution polymerization, suspension polymerization, dispersion polymerization, and emulsion polymerization). In an embodiment of the polymerization step, a polymerization initiator is added to the mixture obtained in the mixing step or to the monomer obtained in the separation step in the presence or absence of a solvent. A preferred polymerization method is solution polymerization, which can produce a highly concentrated solution of a fluorine-containing polymer (P), thereby giving a high yield and being advantageous in the formation of thick films and purification. Thus, the fluorine-containing polymer (P) is preferably a polymer produced according to solution polymerization, and more preferably a polymer produced according to solution polymerization in which a monomer is polymerized in the presence of an aprotic solvent.

In the solution polymerization, the solvent for use is preferably an aprotic solvent. The amount of the aprotic solvent for use in producing a fluorine-containing polymer (P) may be, for example, 80 mass % or lower, lower than 80 mass8, 75 mass % or lower, 70 mass % or lower, 35 mass % or higher and 95 mass % or lower, 35 mass % or higher and 90 mass % or lower, 35 mass % or higher and 80 mass % or lower, 35 mass % or higher and 70 mass % or lower, 35 mass % or higher and lower than 70 mass %, or 60 mass % or higher and 80 mass % or lower, preferably 35 mass % or higher and lower than 80 mass %, more preferably 40 mass % or higher and 75 mass % or lower, and particularly preferably 50 mass % or higher and 70 mass % or lower.

The aprotic solvent is, for example, at least one selected from the group consisting of perfluoroaromatic compounds, perfluorotrialkylamines, perfluoroalkanes, hydrofluorocarbons, perfluorocyclic ethers, hydrofluoroethers, and olefin compounds containing at least one chlorine atom.

The perfluoroaromatic compound is, for example, a perfluoroaromatic compound optionally having one or more perfluoroalkyl groups. The aromatic ring of the perfluoroaromatic compound may be at least one ring selected from the group consisting of a benzene ring, a naphthalene ring, and an anthracene ring. The perfluoroaromatic compound may have one or more (e.g., one, two, or three) aromatic rings.

The perfluoroalkyl group as a substituent is, for example, linear or branched C1-C6, C1-C5, or C1-C4 perfluoroalkyl, preferably linear or branched C1-C3 perfluoroalkyl.

The number of substituents is, for example, one to four, preferably one to three, and more preferably one to two. When a plurality of substituents are present, they may be the same or different.

Examples of perfluoroaromatic compounds include perfluorobenzene, perfluorotoluene, perfluoroxylene, and perfluoronaphthalene.

Preferred examples of perfluoroaromatic compounds include perfluorobenzene and perfluorotoluene.

The perfluorotrialkylamine is, for example, an amine substituted with three linear or branched perfluoroalkyl groups. The number of carbon atoms of each perfluoroalkyl group is, for example, 1 to 10, preferably 1 to 5, and more preferably 1 to 4. The perfluoroalkyl groups may be the same or different, and are preferably the same.

Examples of perfluorotrialkylamines include perfluorotrimethylamine, perfluorotriethylamine, perfluorotripropylamine, perfluorotriisopropylamine, perfluorotributylamine, perfluorotri-sec-butylamine, perfluorotri-tert-butylamine, perfluorotripentylamine, perfluorotriisopentylamine, and perfluorotrineopentylamine.

Preferred examples of perfluorotrialkylamines include perfluorotripropylamine and perfluorotributylamine.

The perfluoroalkane is, for example, a linear, branched, or cyclic C3-C12 (preferably C3-C10, more preferably C3-C6) perfluoroalkane.

Examples of perfluoroalkanes include perfluoropentane, perfluoro-2-methylpentane, perfluorohexane, perfluoro-2-methylhexane, perfluoroheptane, perfluorooctane, perfluorononane, perfluorodecane, perfluorocyclohexane, perfluoro(methylcyclohexane), perfluoro(dimethylcyclohexane) (e.g., perfluoro(1,3-dimethylcyclohexane)), and perfluorodecalin.

Preferred examples of perfluoroalkanes include perfluoropentane, perfluorohexane, perfluoroheptane, and perfluorooctane.

The hydrofluorocarbon is, for example, a C3-C₈ hydrofluorocarbon. Examples of hydrofluorocarbons include CF₃CH₂CF₂H, CF₃CH₂CF₂CH₃, CF₃CHFCHFC₂F₅, 1,1,2,2,3,3,4-heptafluorocyclopentane, CF₃CF₂CF₂CF₂CH₂CH₃, CF₃CF₂CF₂CF₂CF₂CHF₂, and CF₃CF₂CF₂CF₂CF₂CF₂CH₂CH₃.

Preferred examples of hydrofluorocarbons include CF₃CH₂CF₂H, CF₃CH₂CF₂CH₃, and CF₃CHCFCHFC₂F₅.

The perfluorocyclic ether is, for example, a perfluorocyclic ether optionally having one or more perfluoroalkyl groups. The ring of the perfluorocyclic ether may be a 3- to 6-membered ring. The ring of the perfluorocyclic ether may have one or more oxygen atoms as a ring-constituting atom. The ring preferably has one or two oxygen atoms, and more preferably one oxygen atom.

The perfluoroalkyl group as a substituent is, for example, linear or branched C1-C6 perfluoroalkyl, C1-C5 perfluoroalkyl, or C1-C4 perfluoroalkyl. The perfluoroalkyl group is preferably linear or branched Ci-C3 perfluoroalkyl.

The number of substituents is, for example, one to four, preferably one to three, and more preferably one to two.

When a plurality of substituents are present, they may be the same or different.

Examples of perfluorocyclic ethers include perfluorotetrahydrofuran, perfluoro-5-methyltetrahydrofuran, perfluoro-5-ethyltetrahydrofuran, perfluoro-5-propyltetrahydrofuran, perfluoro-5-butyltetrahydrofuran, and perfluorotetrahydropyran.

Preferred examples of perfluorocyclic ethers include perfluoro-5-ethyltetrahydrofuran and perfluoro-5-butyltetrahydrofuran.

The hydrofluoroether is, for example, a fluorine-containing ether.

The hydrofluoroether preferably has a global warming potential (GWP) of 400 or less, and more preferably 300 or less.

Examples of hydrofluoroethers include CF₃CF₂CF₂CF₂OCH₃, CF₃CF₂CF(CF₃)OCH₃, CF₃CF(CF₃)CF₂OCH₃, CF₃CF₂CF₂CF₂O₂H₅, CF₃CH₂OCF₂CHF₂, C₂F₅CF(OCH₃)C₃F₇, (CF₃)₂CHOCH₃, (CF₃)₂CFOCH₃, CHF₂CF₂OCH₂CF₃, CHF₂CF₂CH₂OCF₂CHF₂, CF₃CHFCF₂OCH₃, CF₃CHFCF₂OCF₃, trifluoromethyl 1,2,2,2-tetrafluoroethyl ether (HFE-227me), difluoromethyl 1,1,2,2,2-pentafluoroethyl ether (HFE-227mc), trifluoromethyl 1,1,2,2-tetrafluoroethyl ether (HFE-227pc), difluoromethyl 2,2,2-trifluoroethyl ether (HFE-245mf), 2,2-difluoroethyl trifluoromethyl ether (HFE-245 pf), 1,1,2,3,3,3-hexafluoropropyl methyl ether (CF₃CHFCF₂OCH₃), 1,1,2,2-tetrafluoroethyl 2,2,2-trifluoroethyl ether (CHF₂CF₂OCH₂CF₃), and 1,1,1,3,3,3-hexafluoro-2-methoxy propane ((CF₃)₂CHOCH₃).

Preferred examples of hydrofluoroethers include CF₃CF₂CF₂CF₂OCH₃, CF₃CF₂CF₂CF₂OCH₅, CF₃CH₂OCF₂CHF₂, and C₂F₅CF(OCH₃)C₃F₇. The hydrofluoroether is preferably a compound represented by the following formula (E1):

R²¹—O—R²² (E1)

wherein R²¹ represents linear or branched perfluorobutyl, and R²² represents methyl or ethyl.

The olefin compound containing at least one chlorine atom is a C2-C4 (preferably C2-C3) olefin compound containing at least one chlorine atom in its structure. The olefin compound containing at least one chlorine atom is a compound in which at least one of the hydrogen atoms bonded to the carbon atoms in a C2-C4 hydrocarbon having one or two (preferably one) carbon-carbon double bonds (C═C) is replaced by a chlorine atom. The olefin compound containing at least one chlorine atom is preferably a compound in which at least one of the hydrogen atoms bonded to the two carbon atoms of a carbon-carbon double bond in a C2-C4 hydrocarbon is replaced by a chlorine atom.

The number of chlorine atoms is one to the maximum substitutable number. The number of chlorine atoms may be, for example, one, two, three, four, or five.

The olefin compound containing at least one chlorine atom may contain at least one (e.g., one, two, three, four, or five) fluorine atom.

Examples of olefin compounds containing at least one chlorine atom include CH₂═CHCl, CHCl═CHCl, CCl₂═CHCl, CCl₂═CCl₂, CF₃CH═CHCl, CHF₂CF═CHCl, CFH₂CF═CHCl, CF₃CCl═CFCl, CF₂HCl═CFCl, and CFH₂Cl═CFCl.

Preferred examples of olefin compounds containing at least one chlorine atom include CHCl═CHCl, CHF₂CF═CHCl, CF₃CH═CHCl, and CF₃CCl═CFCl.

The aprotic solvent is preferably a hydrofluoroether from the viewpoint of its smaller environmental impact during use and its ability to dissolve a polymer at a high concentration.

Preferred examples of polymerization initiators for use in the production of the fluorine-containing polymer include di-n-propyl peroxydicarbonate, diisopropyl peroxydicarbonate, diisobutyryl peroxide, di(ω-hydro-dodecafluoroheptanoyl)peroxide, di(ω-hydro-hexadecafluorononanoyl)peroxide, ω-hydro-dodecafluoroheptanoyl-ω-hydro-hexadecafluorononanoyl-peroxide, benzoyl peroxide, tert-butyl peroxypivalate, tert-hexyl peroxypivalate, ammonium persulfate, sodium persulfate, and potassium persulfate.

More preferred examples of polymerization initiators include di-n-propyl peroxydicarbonate, diisopropyl peroxydicarbonate, diisobutyryl peroxide, di(ω-hydro-dodecafluoroheptanoyl)peroxide, benzoyl peroxide, tert-butyl peroxypivalate, tert-hexyl peroxypivalate, and ammonium persulfate.

The amount of the polymerization initiator for use in polymerization reaction may be, for example, 0.0001 g or more and 0.05 g or less, preferably 0.0001 g or more and 0.01 g or less, and more preferably 0.0005 g or more and 0.008 g or less, per gram of all monomers to be subjected to the reaction.

The temperature of the polymerization reaction may be, for example, −10° C. or higher and 160° C. or lower, preferably 0° C. or higher and 160° C. or lower, and more preferably 0° C. or higher and 100° C. or lower.

The reaction time of the polymerization reaction may be preferably 0.5 hours or more and 72 hours or less, more preferably 1 hour or more and 48 hours or less, and even more preferably 3 hours or more and 30 hours or less.

The polymerization reaction may be performed in the presence or absence of an inert gas (e.g., nitrogen gas), and preferably in the presence of an inert gas.

The polymerization reaction may be performed under reduced pressure, atmospheric pressure, or increased pressure.

The polymerization reaction can be performed by adding a monomer to an aprotic solvent containing a polymerization initiator and then subjecting the mixture to polymerization conditions. The polymerization reaction can also be performed by adding a polymerization initiator to an aprotic solvent containing a monomer and then subjecting the mixture to polymerization conditions.

### Method for Producing Fluorine-Containing Monomer (M)

One embodiment of the present disclosure relates to a method for producing a fluorine-containing monomer (M) from a mixture containing the fluorine-containing monomer (M) and a polycyclic aromatic compound (B), including the separation step of separating the polycyclic aromatic compound (B) from the mixture. The method for producing a fluorine-containing monomer (M) of the present disclosure uses a mixture containing a fluorine-containing monomer (M) and a polycyclic aromatic compound (B) as a starting material, and enables the production of the fluorine-containing monomer (M) by removing the polycyclic aromatic compound (B) from the mixture in a simple manner.

In the method for producing a fluorine-containing monomer (M) of the present disclosure, the mixture containing the fluorine-containing monomer (M) and the polycyclic aromatic compound (B) may be the composition of the present disclosure described above. In the production method, the fluorine-containing monomer (M) and the polycyclic aromatic compound (B) are the same as the fluorine-containing monomer (M) and the polycyclic aromatic compound (B) in the composition. However, unless otherwise specified, the polycyclic aromatic compound (B) in the production method also includes a compound in which R¹⁷ is a tert-butyl group, and R¹¹, R¹², R¹³, R¹⁴, R¹⁵, R¹⁶, and R¹⁸ are a hydrogen atom in formula (B1) (i.e., 2-(tert-butyl)anthracene-9,10-dione). In the production method, the polycyclic aromatic compound (B) may exclude a compound in which R¹⁷ is a tert-butyl group, and R¹¹, R¹², R¹³, R¹⁴, R¹⁵, R¹⁶, and R¹⁸ are a hydrogen atom in formula (B1) (i.e., 2-(tert-butyl)anthracene-9,10-dione). Additionally, the mixture in the production method may also contain the other components described above (e.g., a phenol compound (C) and a naphthoquinone compound (D)) in addition to the fluorine-containing monomer (M) and the polycyclic aromatic compound (B). Thus, the above descriptions regarding the fluorine-containing monomer (M), the polycyclic aromatic compound (B), and other components for the composition and the stabilization method are applicable to the method for producing the fluorine-containing monomer (M) unless otherwise specified.

The content of the fluorine-containing monomer (M) in the mixture containing the fluorine-containing monomer (M) and the polycyclic aromatic compound (B) is, for example, 10 to 99.999 mass %, 90 to 99.999 mass %, 92 to 99.999 mass %, 94 to 99.999 mass8, 95 to 99.999 mass8, 96 to 99.999 mass %, 97 to 99.999 mass8, 90 to 99.99 mass8, 92 to 99.99 mass8, 94 to 99.99 mass, 95 to 99.99 mass %, 96 to 99.99 mass8, 97 to 99.99 mass %, 90 to 99.9 mass8, 92 to 99.9 mass, 94 to 99.9 mass %, 95 to 99.9 mass8, 96 to 99.9 mass, 97 to 99.9 mass %, 90 to 99 mass %, 92 to 99 mass8, 94 to 99 mass8, 95 to 99 mass8, 96 to 99 mass %, or 97 to 99 mass %, preferably 50 to 99.999 mass %, and more preferably 90 to 99.999 mass % based on the mass of the mixture.

The content of the polycyclic aromatic compound (B) in the mixture containing the fluorine-containing monomer (M) and the polycyclic aromatic compound (B) is, for example, 0.001 to 90 mass %, 0.001 to 10 mass %, 0.001 to 8 mass %, 0.001 to 6 mass %, 0.001 to 5 mass %, 0.001 to 4 mass %, 0.001 to 3 mass %, 0.01 to 10 mass %, 0.01 to 8 mass %, 0.01 to 6 mass %, 0.01 to 5 mass %, 0.01 to 4 mass %, 0.01 to 3 mass %, 0.1 to 10 mass %, 0.1 to 8 mass %, 0.1 to 6 mass %, 0.1 to 5 mass %, 0.1 to 4 mass %, 0.1 to 3 mass %, 1 to 10 mass %, 1 to 8 mass %, 1 to 6 mass %, 1 to 5 mass %, 1 to 4 mass %, or 1 to 3 mass %, preferably 0.001 to 50 mass %, and more preferably 0.001 to 10 mass %, based on the mass of the mixture.

The mixture containing the fluorine-containing monomer (M) and the polycyclic aromatic compound (B) may contain other components in addition to the fluorine-containing monomer (M) and the polycyclic aromatic compound (B). The content of the other components may be, for example, 0.001 to 8 mass %, 0.001 to 6 mass %, or 0.001 to 5 mass %, based on the mass of the composition.

The method for producing a fluorine-containing monomer (M) includes the separation step of separating the polycyclic aromatic compound (B) from the mixture. This separation step may be performed, for example, in the same manner as in the separation step in the method for producing a fluorine-containing polymer (P) of the present disclosure.

Thus, the separation step of the method for producing a fluorine-containing monomer (M) can use an applicable known method as a method for separating the polycyclic aromatic compound (B). For example, the polycyclic aromatic compound (B) can be separated by adsorption removal using an adsorbent (e.g., silica gel, activated alumina, and zeolite), distillation, filtration, centrifugation etc. In the separation step of the method for producing a fluorine-containing monomer (M), adsorption removal using an adsorbent (e.g., silica gel, activated alumina, and zeolite,) is preferred due to its simplicity in separation compared with distillation.

The present disclosure encompasses, for example, the following aspects.
Item 1.

A composition comprising
a fluorine-containing monomer (M), and
a polycyclic aromatic compound (B),
the fluorine-containing monomer (M) being at least one monomer selected from the group consisting of

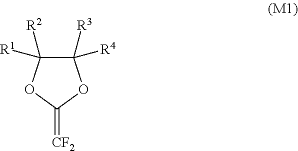

wherein R¹, R², R³, and R⁴ each independently represent a fluorine atom, a perfluoroalkyl group, or a perfluoroalkoxy group,

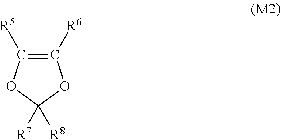

wherein R⁵, R⁶, R⁷, and R⁸ each independently represent a fluorine atom, a perfluoroalkyl group, or a perfluoroalkoxy group, and
a compound represented by formula (M3)

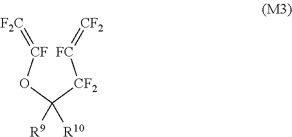

wherein R⁹ and R¹⁰ each independently represent a fluorine atom, a perfluoroalkyl group, or a perfluoroalkoxy group,
   the polycyclic aromatic compound (B) being represented by formula (B1)

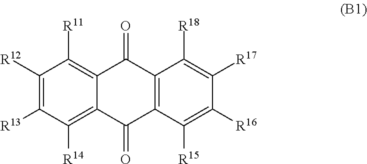

wherein R¹¹, R¹², R¹³, R¹⁴, R¹⁵, R¹⁶, R¹⁷, and R¹⁸ each independently represent a hydrogen atom or an alkyl group, however, excluding an embodiment in which R¹⁷ represents a tert-butyl group, with R¹¹, R¹², R¹³, R¹⁴, R¹⁵, R¹⁶, and R¹⁸ being a hydrogen atom.
Item 2.

The composition according to Item 1, wherein the fluorine-containing monomer (M) is at least one compound selected from the group consisting of a compound represented by the following formula (M1-1), a compound represented by the following formula (M1-2), a compound represented by the following formula (M2-1), a compound represented by the following formula (M2-2), and a compound represented by the following formula (M3-1):

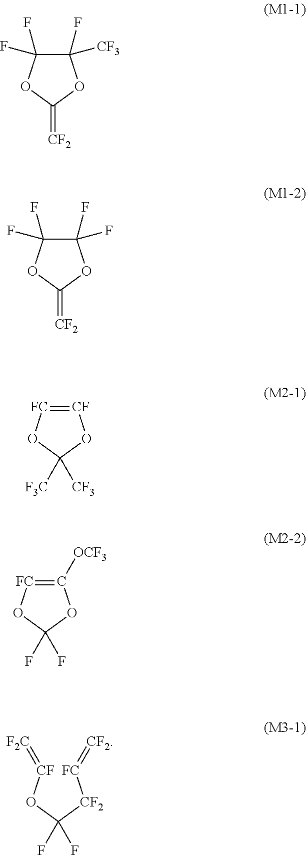

Item 3.

The composition according to Item 1 or 2, wherein R¹¹, R¹², R¹³, R¹⁴, R¹⁵, R¹⁶, R¹⁷, and R¹⁸ each independently represent a hydrogen atom or a C1-C5 alkyl group.
Item 4.

The composition according to any one of Items 1 to 3, wherein the polycyclic aromatic compound (B) is either a compound represented by the following formula (B1-1) or a compound represented by the following formula (B1-2), or both:

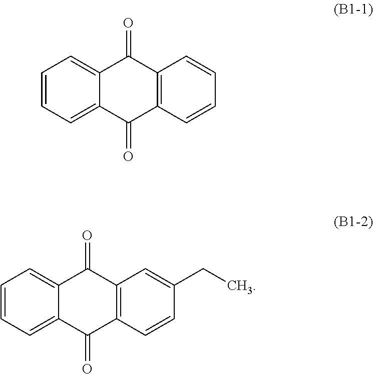 [SMILES: O=C1c2ccccc2C(=O)c2ccccc21 ; CCc1ccc2c(c1)C(=O)c1ccccc1C2=O]

Item 5.

The composition according to any one of Items 1 to 4, wherein the content of the fluorine-containing monomer (M) is 50 to 99.999 mass % based on the mass of the composition, and the content of the polycyclic aromatic compound (B) is 0.001 to 50 mass % based on the mass of the composition.
Item 6.

The composition according to any one of Items 1 to 4, wherein the content of the fluorine-containing monomer (M) is 90 to 99.999 mass based on the mass of the composition, and the content of the polycyclic aromatic compound (B) is 0.001 to 10 mass % based on the mass of the composition.
Item 7.

The composition according to any one of Items 1 to 6, further comprising at least one compound selected from the group consisting of
a phenol compound (C) represented by formula (C1)

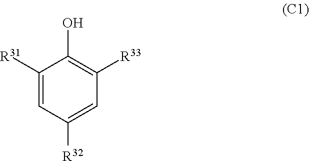

wherein R³¹, R³², and R³³ each independently represent a hydrogen atom, a C1-C5 alkyl group, or a C1-C5 alkoxy group, and
a naphthoquinone compound (D) represented by formula (D1)

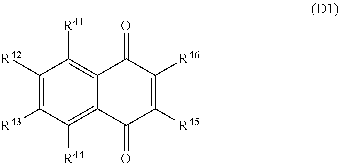

wherein R⁴¹, R⁴², R⁴³, R⁴⁴, R⁴⁵, and R⁴⁶ each independently represent a hydrogen atom or a C1-C5 alkyl group.
Item 8.

A method for stabilizing a fluorine-containing monomer (M), comprising mixing the fluorine-containing monomer (M) and a polycyclic aromatic compound (B), and keeping the temperature of the obtained mixture at 10° C. or lower,
the fluorine-containing monomer (M) being at least one monomer selected from the group consisting of
   a compound represented by formula (M1)

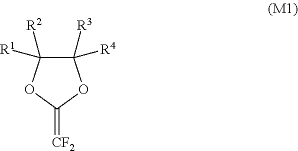

wherein R¹, R², R³, and R⁴ each independently represent a fluorine atom, a perfluoroalkyl group, or a perfluoroalkoxy group,
a compound represented by formula (M2)

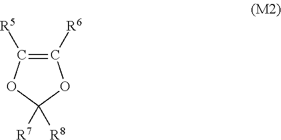

wherein R⁵, R⁶, R⁷, and R⁸ each independently represent a fluorine atom, a perfluoroalkyl group, or a perfluoroalkoxy group, and
a compound represented by formula (M3)

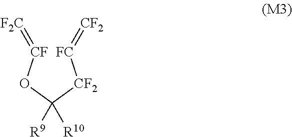

wherein R⁹ and R¹⁰ each independently represent a fluorine atom, a perfluoroalkyl group, or a perfluoroalkoxy group,
   the polycyclic aromatic compound (B) being represented by formula (B1)

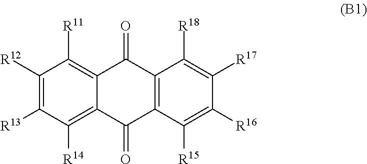

wherein R¹¹, R¹², R¹³, R¹⁴, R¹⁵, R¹⁶, R¹⁷, and R¹⁸ each independently represent a hydrogen atom or an alkyl group.
Item 9.

The method for stabilizing a fluorine-containing monomer (M) according to Item 8, wherein the fluorine-containing monomer (M) is at least one compound selected from the group consisting of a compound represented by the following formula (M1-1), a compound represented by the following formula (M1-2), a compound represented by the following formula (M2-1), a compound represented by the following formula (M2-2), and a compound represented by the following formula (M3-1):

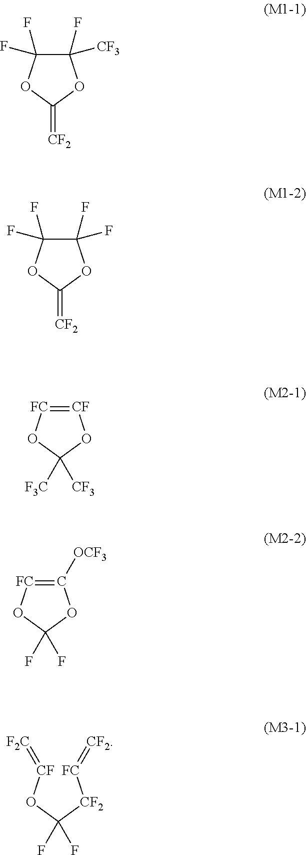

Item 10.

The method for stabilizing a fluorine-containing monomer (M) according to Item 8 or 9, wherein R¹¹, R¹², R¹³, R¹⁴, R¹⁵, R¹⁶, R¹⁷, and R¹⁸ each independently represent a hydrogen atom or a C1-C5 alkyl group.
Item 11.

The method for stabilizing a fluorine-containing monomer (M) according to any one of Items 8 to 10, wherein the polycyclic aromatic compound (B) is either a compound represented by the following formula (B1-1) or a compound represented by the following formula (B1-2), or both:

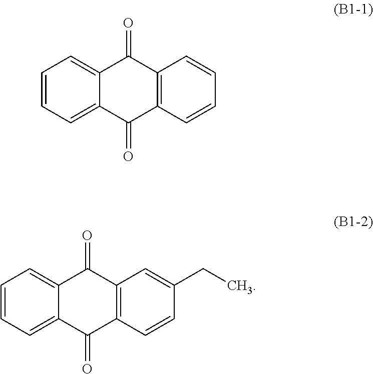 [SMILES: O=C1c2ccccc2C(=O)c2ccccc21 ; CCc1ccc2c(c1)C(=O)c1ccccc1C2=O]

Item 12.

The method for stabilizing a fluorine-containing monomer (M) according to any one of Items 8 to 11, wherein the proportion of the polycyclic aromatic compound (B) to be mixed is 0.001 to 12 mass % based on the mass of the fluorine-containing monomer (M) taken as 100.
Item 13.

The method for stabilizing a fluorine-containing monomer (M) according to any one of Items 8 to 12, comprising further adding at least one compound selected from the group consisting of
a phenol compound (C) represented by formula (C1)

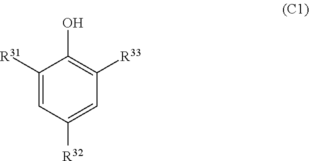

wherein R³¹, R³², and R³³ each independently represent a hydrogen atom, a C1-C5 alkyl group, or a C1-C5 alkoxy group, and
a naphthoquinone compound (D) represented by formula (D1)

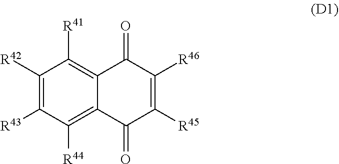

wherein R⁴¹, R⁴², R⁴³, R⁴⁴, R⁴⁵, and R⁴⁶ each independently represent a hydrogen atom or a C1-C5 alkyl group, when mixing the fluorine-containing monomer (M) and the polycyclic aromatic compound (B).
Item 14.

A method for producing a fluorine-containing polymer (P) by polymerizing a fluorine-containing monomer (M), comprising mixing the fluorine-containing monomer (M) and a polycyclic aromatic compound (B),
the fluorine-containing monomer (M) being at least one monomer selected from the group consisting of
   a compound represented by formula (M1)

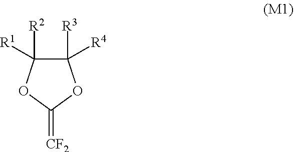

wherein R¹, R², R³, and R⁴ each independently represent a fluorine atom, a perfluoroalkyl group, or a perfluoroalkoxy group,
a compound represented by formula (M2)

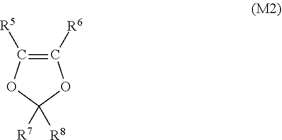

wherein R⁵, R⁶, R⁷, and R⁸ each independently represent a fluorine atom, a perfluoroalkyl group, or a perfluoroalkoxy group, and
a compound represented by formula (M3)

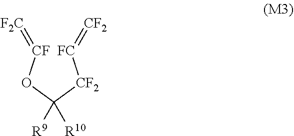

wherein R⁹ and R¹⁰ each independently represent a fluorine atom, a perfluoroalkyl group, or a perfluoroalkoxy group,
   the polycyclic aromatic compound (B) being represented by formula (B1)

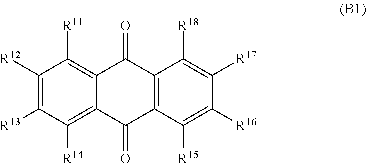

wherein R¹¹, R¹², R¹³, R¹⁴, R¹⁵, R¹⁶, R¹⁷, and R¹⁸ each independently represent a hydrogen atom or an alkyl group.
Item 15.

The method for producing a fluorine-containing polymer (P) according to Item 14, further comprising separating the polycyclic aromatic compound (B) from the mixture containing the fluorine-containing monomer (M) and the polycyclic aromatic compound (B) obtained in the mixing step.
Item 16.

The method for producing a fluorine-containing polymer (P) according to Item 14 or 15, wherein the fluorine-containing monomer (M) is at least one compound selected from the group consisting of a compound represented by the following formula (M1-1), a compound represented by the following formula (M1-2), a compound represented by the following formula (M2-1), a compound represented by the following formula (M2-2), and a compound represented by the following formula (M3-1):

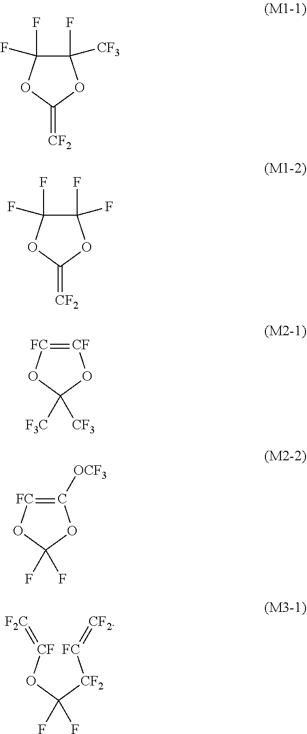

Item 17.

The method for producing a fluorine-containing polymer (P) according to any one of Items 14 to 16, wherein R¹¹, R¹², R¹³, R¹⁴, R¹⁵, R¹⁶, R¹⁷, and R¹⁸ each independently represent a hydrogen atom or a C1-C5 alkyl group.
Item 18.

The method for producing a fluorine-containing polymer (P) according to any one of Items 14 to 17, wherein the polycyclic aromatic compound (B) is either a compound represented by the following formula (B1-1) or a compound represented by the following formula (B1-2), or both:

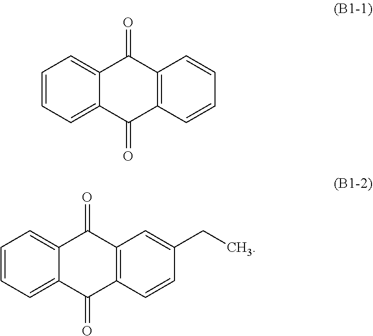 [SMILES: O=C1c2ccccc2C(=O)c2ccccc21 ; CCc1ccc2c(c1)C(=O)c1ccccc1C2=O]

Item 19.

The method for producing a fluorine-containing polymer (P) according to any one of Items 14 to 18, wherein the proportion of the polycyclic aromatic compound (B) to be mixed in the mixing step is 0.001 to 12 mass % based on the mass of the fluorine-containing monomer (M) taken as 100.
Item 20.

The method for producing a fluorine-containing polymer (P) according to any one of Items 14 to 19, comprising further adding at least one compound selected from the group consisting of
a phenol compound (C) represented by formula (C1)

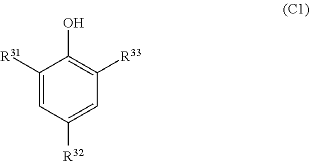

wherein R³¹, R³², and R³³ each independently represent a hydrogen atom, a C1-C5 alkyl group, or a C1-C5 alkoxy group, and
a naphthoquinone compound (D) represented by formula (D1)

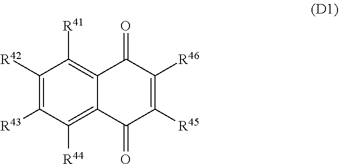

wherein R⁴¹, R⁴², R⁴³, R⁴⁴, R⁴⁵, and R⁴⁶ each independently represent a hydrogen atom or a C1-C5 alkyl group,
in the mixing step of mixing the fluorine-containing monomer (M) and the polycyclic aromatic compound (B).
Item 21.

A method for producing a fluorine-containing monomer (M) from a mixture containing the fluorine-containing monomer (M) and a polycyclic aromatic compound (B), comprising separating the polycyclic aromatic compound (B) from the mixture,
the fluorine-containing monomer (M) being at least one monomer selected from the group consisting of
   a compound represented by formula (M1)

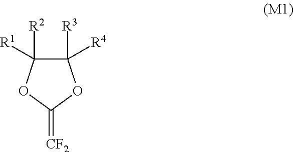

wherein R¹, R², R³, and R⁴ each independently represent a fluorine atom, a perfluoroalkyl group, or a perfluoroalkoxy group,
a compound represented by formula (M2)

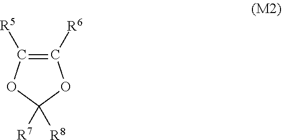

wherein R⁵, R⁶, R⁷, and R⁸ each independently represent a fluorine atom, a perfluoroalkyl group, or a perfluoroalkoxy group, and
   a compound represented by formula (M3)

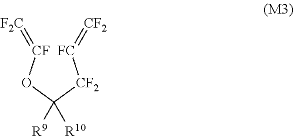

wherein R⁹ and R¹⁰ each independently represent a fluorine atom, a perfluoroalkyl group, or a perfluoroalkoxy group,
   the polycyclic aromatic compound (B) being represented by formula (B1)

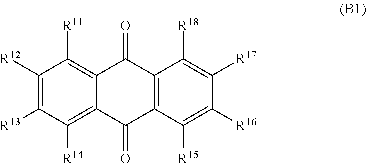

wherein R¹¹, R¹², R¹³, R¹⁴, R¹⁵, R¹⁶, R¹⁷, and R¹⁸ each independently represent a hydrogen atom or an alkyl group.
Item 22.

The method for producing a fluorine-containing monomer (M) according to Item 21, wherein the fluorine-containing monomer (M) is at least one compound selected from the group consisting of a compound represented by the following formula (M1-1), a compound represented by the following formula (M1-2), a compound represented by the following formula (M2-1), a compound represented by the following formula (M2-2), and a compound represented by the following formula (M3-1):

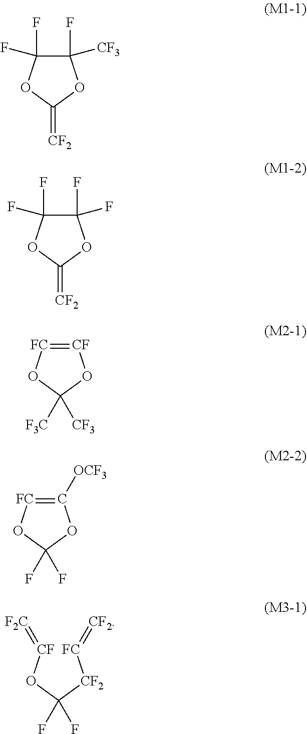

Item 23.

The method for producing a fluorine-containing monomer (M) according to Item 21 or 22, wherein R¹¹, R¹², R¹³, R¹⁴, R¹⁵, R¹⁶, R¹⁷, and R¹⁸ each independently represent a hydrogen atom or a C1-C5 alkyl group.
Item 24.

The method for producing a fluorine-containing monomer (M) according to any one of Items 21 to 23, wherein the polycyclic aromatic compound (B) is either a compound represented by the following formula (B1-1) or a compound represented by the following formula (B1-2), or both:

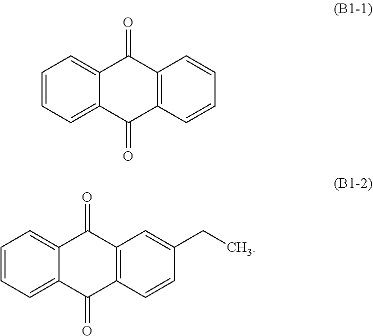 [SMILES: O=C1c2ccccc2C(=O)c2ccccc21 ; CCc1ccc2c(c1)C(=O)c1ccccc1C2=O]

Item 25.

The method for producing a fluorine-containing monomer (M) according to any one of Items 21 to 24, wherein the proportion of the polycyclic aromatic compound (B) in the mixture is 0.001 to 12 mass % based on the mass of the fluorine-containing monomer (M) taken as 100.

### EXAMPLES

The following describes an embodiment of the present disclosure in more detail with reference to Examples. However, the present disclosure is not limited to the Examples.

The fluorine-containing monomer and the substances that were allowed to be present with the monomer in the following Examples are listed below.

### Fluorine-Containing Monomer

### Perfluoro(2-methylene-4-methyl-1,3-dioxolane) (monomer (M1-1))

### Substances Allowed to be Present With Fluorine-Containing Monomer

2-ethylanthraquinone (compound (B1-2))
2,6-di-t-butyl-p-cresol (compound (C1-1))
Phenothiazine
4-t-butylcatechol
1 4-naphthoquinone (compound (D1-1))
Tetrafluorohydroquinone

### Monomer Quantification

To the monomer (M1-1), 1 mass % of perfluorohexane was added, and the content of the monomer (M1-1) was determined on a relative basis from the GC area ratio of the monomer (M1-1) to the perfluorohexane. The value was used as a quantitative value.

### Example 1

5.0 g (quantitative value: 95.0%) of the monomer (M1-1) and 5.0 mg of the compound (B1-2) were placed in a 50 mL plastic container, shaken, and mixed, and then stored in a freezer set to −20° C. for 2 days. The quantitative value after storage was 94.6%.

### Comparative Example 1

A test was performed in the same manner as in Example 1, except that 5.0 mg (1000 ppm) of 2,6-di-t-butyl-p-cresol was used instead of the compound (B1-2). The quantitative value after storage was 94.0%.

### Comparative Example 2

A test was performed in the same manner as in Example 1, except that 5.0 mg (1000 ppm) of phenothiazine was used instead of the compound (B1-2). The quantitative value after storage was 93.9%.

### Comparative Example 3

A test was performed in the same manner as in Example 1, except that 5.0 mg (1000 ppm) of 4-t-butylcatechol was used instead of the compound (B1-2). The quantitative value after storage was 93.6%.

### Example 2

5.0 g (quantitative value: 93.1%) of the monomer (M1-1) and 5.0 mg of the compound (B1-2) were placed in a 50 mL plastic container, shaken, and mixed, and then stored in a freezer set to 0° C. for 2 days. The quantitative value after storage was 89.7%.

### Comparative Example 4

A test was performed in the same manner as in Example 2, except that 5.0 mg (1000 ppm) of 1, 4-naphthoquinone was used instead of the compound (B1-2). The quantitative value after storage was 88.9%.

### Comparative Example 5

A test was performed in the same manner as in Example 2, except that 5.0 mg (1000 ppm) of tetrafluorohydroquinone was used instead of the compound (B1-2). The quantitative value after storage was 87.8%.

### Example 3

10.0 g (quantitative value: 96.1%) of the monomer (M1-1) was placed in a 50 mL plastic container, and then 10.0 mg (1000 ppm) of the compound (B1-2) was added. The sample bottle was sealed and shaken to mix the components. The contents in the bottle were subjected to adsorption treatment using 0.10 g (1 mass %) of silica gel (Wakogel C-200, Fujifilm Wako Pure Chemical Corporation) to obtain a treated material. No solid residue was found in the treated material, and quantitative analysis by liquid chromatography (LC) according to a calibration curve method indicated that the concentration of the compound (B1-2) in the treated material was less than 1 ppm. The compound (B1-2) was confirmed to be easily separated from the monomer (M1-1).

### Comparative Example 6

A test was performed in the same manner as in Example 3, except that 10.0 mg (1000 ppm) of 2, 6-di-t-butyl-p-cresol was used instead of the compound (B1-2). The concentration of 2,6-di-t-butyl-p-cresol in the treated material was 510 ppm.

### Example 4

To a 50 mL glass vessel, 10 g of the monomer (M1-1) obtained according to the method in Example 3, 15 g of methyl nonafluorobutyl ether (solvent), and 0.017 g of a solution of 50 mass % di-n-propyl peroxydicarbonate in methanol were added, and a polymerization reaction was allowed to proceed for 20 hours with heating so that the internal temperature reached 40° C. Thereafter, 20 g of methanol was added to the obtained 10 polymerization solution, and the resulting precipitate was collected by filtration. The filtrate was washed with 100 g of methanol and then dried at 120° C. for 24 hours, thereby obtaining 7.9 g of a fluorine-containing polymer formed of solid units (U1-1).

1. A composition comprising
a fluorine-containing monomer (M), and
a polycyclic aromatic compound (B),
the fluorine-containing monomer (M) being at least one monomer selected from the group consisting of a compound represented by formula (MI) 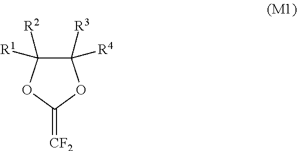
wherein R¹, R², R³, and R⁴ each independently represent a fluorine atom, a perfluoroalkyl group, or a perfluoroalkoxy group,
a compound represented by formula (M2) 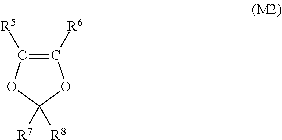
wherein R⁵, R⁶, R⁷, and R⁸ each independently represent a fluorine atom, a perfluoroalkyl group, or a perfluoroalkoxy group, and
a compound represented by formula (M3) 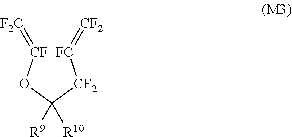
wherein R⁹ and R¹⁰ each independently represent a fluorine atom, a perfluoroalkyl group, or a perfluoroalkoxy group,
the polycyclic aromatic compound (B) being represented by formula (B1) 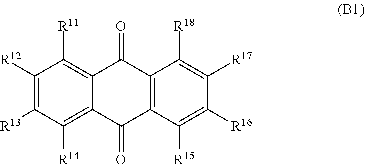
wherein R¹¹, R¹², R¹³, R¹⁴, R¹⁵, R¹⁶, R¹⁷, and R¹⁸ each independently represent a hydrogen atom or an alkyl group, however, excluding an embodiment in which R¹⁷ represents a tert-butyl group, with R¹, R¹², R¹³, R¹⁴, R¹⁵, R¹⁶, and R¹⁸ being a hydrogen atom.

2. The composition according to claim 1, wherein the fluorine-containing monomer (M) is at least one compound selected from the group consisting of a compound represented by the following formula (M1-1), a compound represented by the following formula (M1-2), a compound represented by the following formula (M2-1), a compound represented by the following formula (M2-2), and a compound represented by the following formula (M3-1):

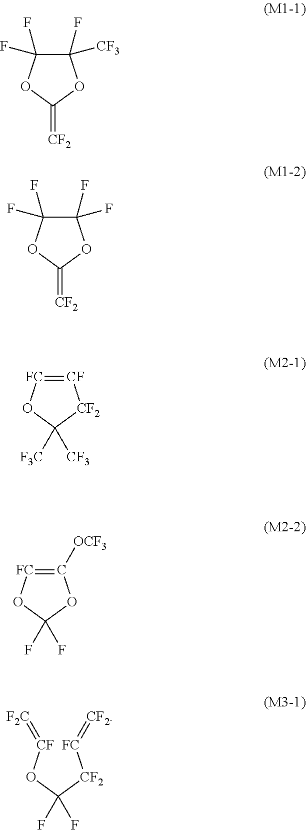

3. The composition according to claim 1, wherein R¹¹, R¹², R¹³, R¹⁴, R¹⁵, R 16, R¹⁷, and R¹⁸ each independently represent a hydrogen atom or a C1-C5 alkyl group.

4. The composition according to claim 1, wherein the polycyclic aromatic compound (B) is either a compound represented by the following formula (B1-1) or a compound represented by the following formula (B1-2), or both:

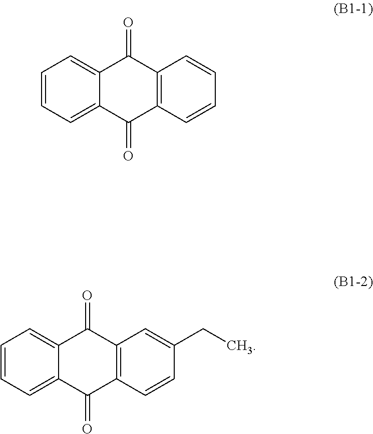 [SMILES: O=C1c2ccccc2C(=O)c2ccccc21 ; CCc1ccc2c(c1)C(=O)c1ccccc1C2=O]

5. The composition according to claim 1, wherein the content of the fluorine-containing monomer (M) is 50 to 99.999 mass % based on the mass of the composition, and the content of the polycyclic aromatic compound (B) is 0.001 to 50 mass % based on the mass of the composition.

6. The composition according to claim 1, wherein the content of the fluorine-containing monomer (M) is 90 to 99.999 mass % based on the mass of the composition, and the content of the polycyclic aromatic compound (B) is 0.001 to 10 mass % based on the mass of the composition.

7. The composition according to claim 1, further comprising at least one compound selected from the group consisting of
a phenol compound (C) represented by formula (C1) 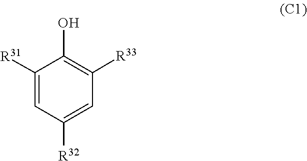
wherein R³¹, R³², and R³³ each independently represent a hydrogen atom, a C1-C5 alkyl group, or a C1-C5 alkoxy group, and
a naphthoquinone compound (D) represented by formula (D1) 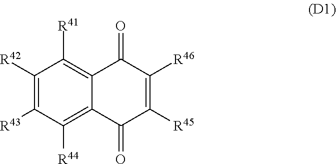
wherein R⁴¹, R⁴², R⁴³, R⁴⁴, R⁴⁵, and R⁴⁶ each independently represent a hydrogen atom or a C1-C5 alkyl group.

8. A method for stabilizing a fluorine-containing monomer (M), comprising mixing the fluorine-containing monomer (M) and a polycyclic aromatic compound (B), and keeping the temperature of the obtained mixture at 10° C. or lower,
the fluorine-containing monomer (M) being at least one monomer selected from the group consisting of
a compound represented by formula (M1) 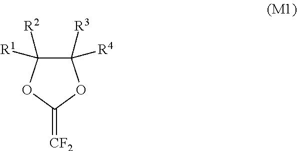
wherein R¹, R², R³, and R⁴ each independently represent a fluorine atom, a perfluoroalkyl group, or a perfluoroalkoxy group,
a compound represented by formula (M2) 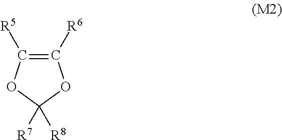
wherein R⁵, R⁶, R⁷, and R⁸ each independently represent a fluorine atom, a perfluoroalkyl group, or a perfluoroalkoxy group, and
a compound represented by formula (M3) 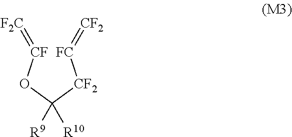
wherein R⁹ and R¹⁰ each independently represent a fluorine atom, a perfluoroalkyl group, or a perfluoroalkoxy group,
the polycyclic aromatic compound (B) being represented by formula (B1) 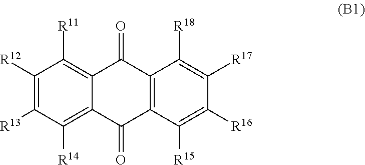
wherein R¹¹, R¹², R¹³, R¹⁴, R¹⁵, R¹⁶, R¹⁷, and R¹⁸ each independently represent a hydrogen atom or an alkyl group.

9. The method for stabilizing a fluorine-containing monomer (M) according to claim 8, wherein the fluorine-containing monomer (M) is at least one compound selected from the group consisting of a compound represented by the following formula (M1-1), a compound represented by the following formula (M1-2), a compound represented by the following formula (M2-1), a compound represented by the following formula (M2-2), and a compound represented by the following formula (M3-1):

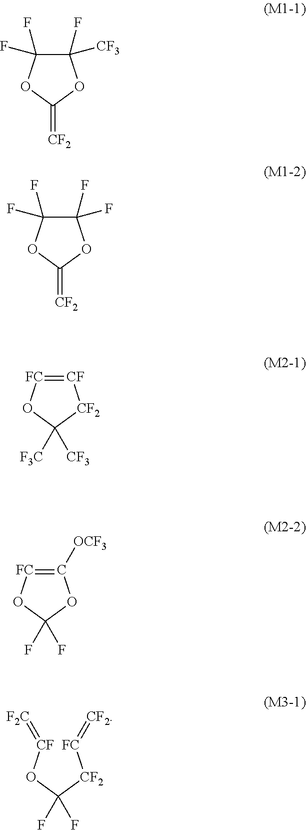

10. The method for stabilizing a fluorine-containing monomer (M) according to claim 8, wherein R¹¹, R¹², R¹³, R¹⁴, R¹⁵, R¹⁶, R¹⁷, and R¹⁸ each independently represent a hydrogen atom or a C1-C5 alkyl group.

11. The method for stabilizing a fluorine-containing monomer (M) according to claim 8, wherein the polycyclic aromatic compound (B) is either a compound represented by the following formula (B1-1) or a compound represented by the following formula (B1-2), or both:

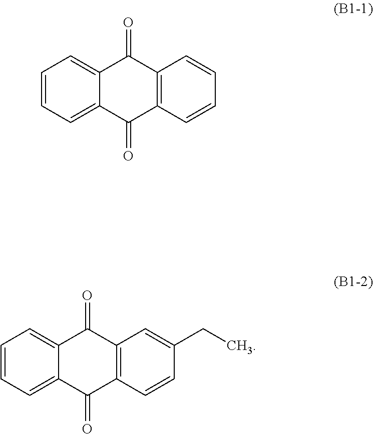 [SMILES: O=C1c2ccccc2C(=O)c2ccccc21 ; CCc1ccc2c(c1)C(=O)c1ccccc1C2=O]

12. The method for stabilizing a fluorine-containing monomer (M) according to claim 8, wherein the proportion of the polycyclic aromatic compound (B) to be mixed is 0.001 to 12 mass % based on the mass of the fluorine-containing monomer (M) taken as 100.

13. The method for stabilizing a fluorine-containing monomer (M) according to claim 8, comprising further adding at least one compound selected from the group consisting of
a phenol compound (C) represented by formula (C1) 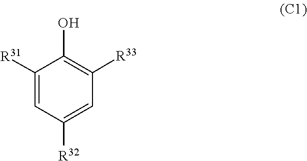
wherein R³¹, R³², and R³³ each independently represent a hydrogen atom, a C1-C5 alkyl group, or a C1-C5 alkoxy group, and
a naphthoquinone compound (D) represented by formula (D1) 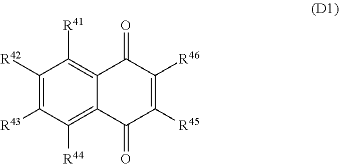
wherein R⁴¹, R⁴², R⁴³, R⁴⁴, R⁴⁵, and R⁴⁶ each independently represent a hydrogen atom or a C1-C5 alkyl group, when mixing the fluorine-containing monomer (M) and the polycyclic aromatic compound (B).

14. A method for producing a fluorine-containing polymer (P) by polymerizing a fluorine-containing monomer (M), comprising mixing the fluorine-containing monomer (M) and a polycyclic aromatic compound (B),
the fluorine-containing monomer (M) being at least one monomer selected from the group consisting of
a compound represented by formula (M1) 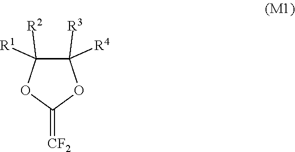
wherein R¹, R², R³, and R⁴ each independently represent a fluorine atom, a perfluoroalkyl group, or a perfluoroalkoxy group,
a compound represented by formula (M2) 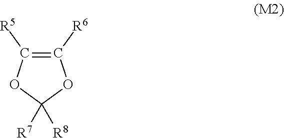
wherein R⁵, R⁶, R⁷, and R⁸ each independently represent a fluorine atom, a perfluoroalkyl group, or a perfluoroalkoxy group, and
a compound represented by formula (M3) 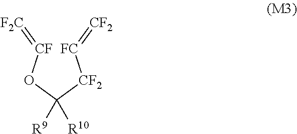
wherein R⁹ and R¹⁰ each independently represent a fluorine atom, a perfluoroalkyl group, or a perfluoroalkoxy group,
the polycyclic aromatic compound (B) being represented by formula (B1) 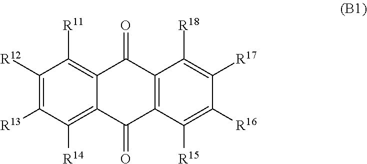
wherein R¹¹, R¹², R¹³, R¹⁴, R¹⁵, R¹⁶, R¹⁷, and R¹⁸ each independently represent a hydrogen atom or an alkyl group.

15. The method for producing a fluorine-containing polymer (P) according to claim 14, further comprising separating the polycyclic aromatic compound (B) from the mixture containing the fluorine-containing monomer (M) and the polycyclic aromatic compound (B) obtained in the mixing step.

16. The method for producing a fluorine-containing polymer (P) according to claim 14, wherein the fluorine-containing monomer (M) is at least one compound selected from the group consisting of a compound represented by the following formula (M1-1), a compound represented by the following formula (M1-2), a compound represented by the following formula (M2-1), a compound represented by the following formula (M2-2), and a compound represented by the following formula (M3-1):

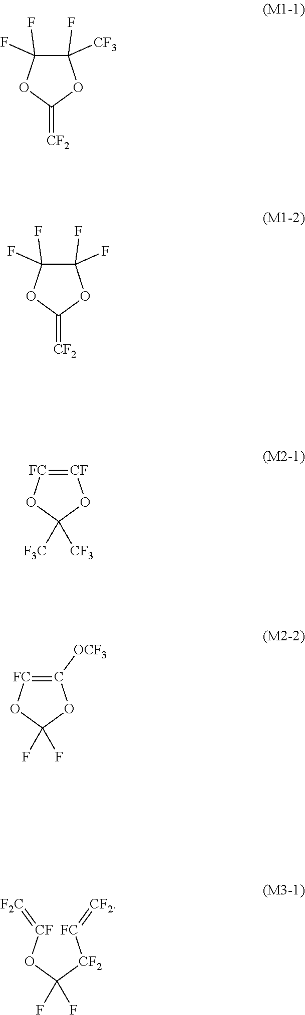

17. The method for producing a fluorine-containing polymer (P) according to claim 14, wherein R¹¹, R¹², R¹³, R¹⁴, R¹⁵, R¹⁶, R¹⁷, and R¹⁸ each independently represent a hydrogen atom or a C1-C5 alkyl group.

18. The method for producing a fluorine-containing polymer (P) according to claim 14, wherein the polycyclic aromatic compound (B) is either a compound represented by the following formula (B1-1) or a compound represented by the following formula (B1-2), or both:

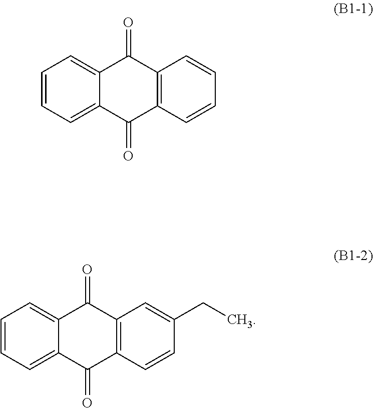 [SMILES: O=C1c2ccccc2C(=O)c2ccccc21 ; CCc1ccc2c(c1)C(=O)c1ccccc1C2=O]

19. The method for producing a fluorine-containing polymer (P) according to claim 14, wherein the proportion of the polycyclic aromatic compound (B) to be mixed in the mixing step is 0.001 to 12 mass % based on the mass of the fluorine-containing monomer (M) taken as 100.

20. The method for producing a fluorine-containing polymer (P) according to claim 14, comprising further adding at least one compound selected from the group consisting of
a phenol compound (C) represented by formula (C1) 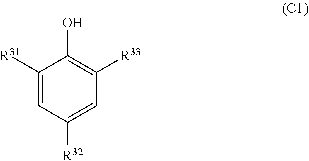
wherein R³¹, R³², and R³³ each independently represent a hydrogen atom, a C1-C5 alkyl group, or a C1-C5 alkoxy group, and
a naphthoquinone compound (D) represented by formula (D1) 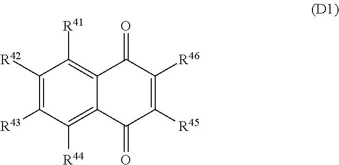
wherein R⁴¹, R⁴², R⁴³, R⁴⁴, R⁴⁵, and R⁴⁶ each independently represent a hydrogen atom or a C1-C5 alkyl group,
in the mixing step of mixing the fluorine-containing monomer (M) and the polycyclic aromatic compound (B).

21. A method for producing a fluorine-containing monomer (M) from a mixture containing the fluorine-containing monomer (M) and a polycyclic aromatic compound (B), comprising separating the polycyclic aromatic compound (B) from the mixture,
the fluorine-containing monomer (M) being at least one monomer selected from the group consisting of
a compound represented by formula (M1) 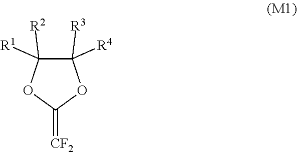
wherein R¹, R², R³, and R⁴ each independently represent a fluorine atom, a perfluoroalkyl group, or a perfluoroalkoxy group,
a compound represented by formula (M2) 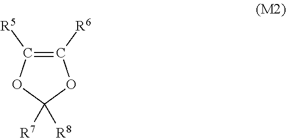
wherein R⁵, R⁶, R⁷, and R⁸ each independently represent a fluorine atom, a perfluoroalkyl group, or a perfluoroalkoxy group, and
a compound represented by formula (M3) 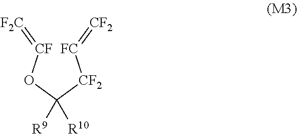
wherein R⁹ and R¹⁰ each independently represent a fluorine atom, a perfluoroalkyl group, or a perfluoroalkoxy group,
the polycyclic aromatic compound (B) being represented by formula (B1) 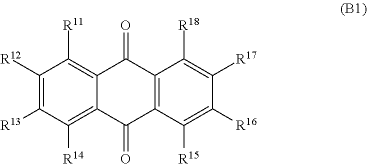
wherein R¹¹, R¹², R¹³, R¹⁴, R¹⁵, R¹⁶, R¹⁷, and R¹⁸ each independently represent a hydrogen atom or an alkyl group.

22. The method for producing a fluorine-containing monomer (M) according to claim 21, wherein the fluorine-containing monomer (M) is at least one compound selected from the group consisting of a compound represented by the following formula (M1-1), a compound represented by the following formula (M1-2), a compound represented by the following formula (M2-1), a compound represented by the following formula (M2-2), and a compound represented by the following formula (M3-1):

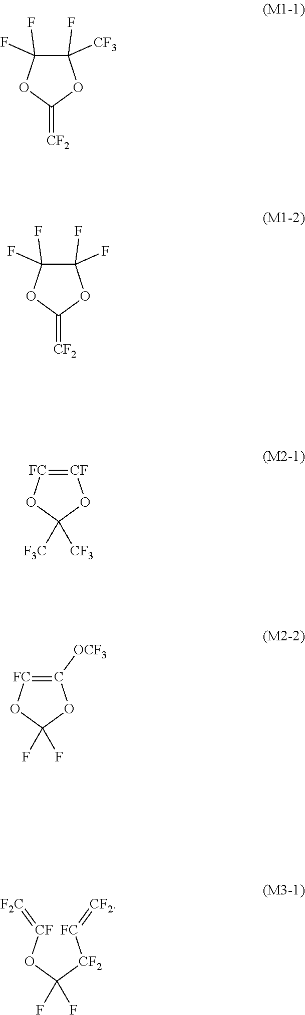

23. The method for producing a fluorine-containing monomer (M) according to claim 21, wherein R¹¹, R¹², R¹³, R¹⁴, R¹⁵, R¹⁶, R¹⁷, and R¹⁸ each independently represent a hydrogen atom or a C1-C5 alkyl group.

24. The method for producing a fluorine-containing monomer (M) according to claim 21, wherein the polycyclic aromatic compound (B) is either a compound represented by the following formula (B1-1) or a compound represented by the following formula (B1-2), or both:

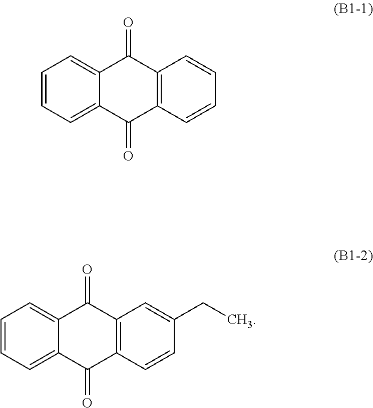 [SMILES: O=C1c2ccccc2C(=O)c2ccccc21 ; CCc1ccc2c(c1)C(=O)c1ccccc1C2=O]

25. The method for producing a fluorine-containing monomer (M) according to claim 21, wherein the proportion of the polycyclic aromatic compound (B) in the mixture is 0.001 to 12 mass % based on the mass of the fluorine-containing monomer (M) taken as 100.
